(19) United States
(12) Patent Application Publication
(10) Pub. No.: US 2003/0032022 A1
(43) Pub. Date: Feb. 13, 2003

(54) Variants of IL-1 beta gene and CD46 gene for diagnosing unexplained recurrent pregnancy loss

(75) Inventors: Joseph A. Hill, Brookline, MA (US); Zhigang C. Wang, West Roxbury, MA (US); Deborah Anderson, Brookline, MA (US); Edmond J. Yunis, Concord, MA (US)

Correspondence Address: Elizabeth R. Plumer, c/o Wolf, Greenfield & Sacks, P.C., Federal Reserve Plaza, 600 Atlantic Avenue, Boston, MA, 02210, US

(73) Assignee: The Brigham and Women's Hospital, Inc., Boston, MA (US)

(21) Appl. No.: 09/950,935

(22) Filed: Sep. 12, 2001

Related U.S. Application Data
(60) Provisional application No. 60/231,785, filed on Sep. 12, 2000.

(51) Int. Cl. C12Q 1/68; G01N 33/53

(52) U.S. Cl. 435/6; 436/510

(57) ABSTRACT

The discovery of an association between two variants of the human interleukin-1 gene (IL1B) beta promoter region and a variant of the CD46 gene intron 1 and T-helper type 1 immunity in unexplained recurrent pregnancy loss (URPL) is disclosed. These two IL1B variants are characterized by a base C at position-511 (IL1B-511C) and a base T at position-31 (IL1B-31T) from the transcriptional start site of the IL1B gene. The CD46 gene intron 1 variant is characterized by a change in the HindIII site in this intron. These IL1B promoter variants and CD46 gene intron 1 variants, and reagents for detecting said variants, are useful as diagnostic markers for the diagnosis and management of recurrent pregnancy loss. Accordingly, the invention provides methods and compositions which identify these variants for determining a subject's propensity for having reproductive failure and, particularly, reproductive failure attributed to Th1 cytokines.

### RELATED APPLICATIONS

This application claims priority under 35 U.S.C. 119 from Provisional U.S. patent Application Serial No. 60/231,785 filed on Sep. 12, 2000, entitled, Variants of IL-1 Beta Gene Promoter Region for Diagnosing Unexplained Recurrent Pregnancy Loss. The contents of the provisional application are hereby expressly incorporated by reference.

### GOVERNMENT SUPPORT

The work was supported, in part, by Grant Number HL29583 from the National Institutes of Health. The government may retain certain rights in this invention.

### FIELD OF THE INVENTION

The present invention relates to unexplained recurrent pregnancy loss and the identification of variants of the IL-1 beta gene promoter and the CD46 gene intron 1 for the diagnosis, prognosis, and therapy associated with this condition, and as research tools to identify other useful agents for these applications. Methods and compositions useful for these applications are disclosed.

### BACKGROUND OF THE INVENTION

Recurrent pregnancy loss (RPL) is a common disorder in early gestation, the cause of which remains unexplained in approximately 50% of cases. Various suggestions have been made for the causes of such pregnancy loss; however, accurate diagnostic tests for identifying women who are prone to RPL have not yet been developed. Accordingly, a need exists to identify genetic markers that are associated with RPL to allow the development of compositions useful for the diagnosis, prognosis and therapy for treating RPL.

### SUMMARY OF THE INVENTION

The invention is based, in part, on the discovery of an association between the variants of the Interleukin-1 beta gene promoter and variants of the CD46 gene intron 1 and unexplained recurrent pregnancy loss. Although not wishing to be bound to any particular theory or mechanism, Applicants believe that such unexplained recurrent pregnancy loss is compositions and methods of the present invention are directed to compositions containing the isolated variants, agents which are useful for their detection (e.g., amplification primers), functional equivalents of the foregoing, and the use of such compositions for diagnosing and/or treating RPL. Thus, the isolated genetic markers (and/or agents which are useful for their detection) for RPL can be incorporated into assay screening kits for use in, for example, hybridization assays, to identify women having a propensity to RPL who carry this genetic marker. The identification of the genetic markers for RPL, therefore, permits a more accurate prediction and diagnosis of this condition then has heretofore been possible. Thus, the invention encompasses compositions, as well as screening assays, diagnostic and therapeutic methods that are useful for research as well as for diagnosing and/or treating RPL.

According to a first aspect of the invention, a method for evaluating a risk of recurrent pregnancy loss in a subject suspected of having immunologic reproductive failure is provided. The method involves: (a) testing a biological sample obtained from the subject for the presence of: (1) a variant in the IL-1 beta promoter region and (2) a variant in the CD46 gene intron 1 region, wherein the presence of the variant in the IL-1B beta promoter region and of the variant in the CD46 gene intron 1 region is indicative of an elevated risk of developing recurrent pregnancy loss. In preferred embodiments, the variant in the IL-1 beta promoter region is a polymorphism of the IL-1 beta gene at position-5 11 and/or a polymorphism of the IL-1 beta gene at position-31 and the variant in the CD46 gene intron 1 region is a polymorphism in the CD46 gene intron 1 region in the HindlIl restriction site.

According to second aspect of the invention, a method for evaluating a risk of recurrent pregnancy loss in a subject suspected of having immunologic reproductive failure is provided which involves: (a) testing a biological sample obtained from the subject for the presence of: (1) a variant in the IL-1 beta promoter region or for (2) the presence of a variant in the CD46 gene intron 1 region, wherein the presence of the variant in the IL-1B beta promoter region and the variant in the CD46 gene intron 1 region is indicative of an elevated risk of developing recurrent pregnancy loss. The preferred embodiments detect the variants disclosed in reference to the first aspect of the invention, namely, a polymorphism of the IL- I beta gene at position-511, a polymorphism of the IL-1 beta gene at position-31, or a polymorphism in the CD46 gene intron 1 region in the HindIII restriction site.

According to a third aspect of the invention, kits useful for evaluating the risk of recurrent pregnancy loss in a subject suspected of having immunologic reproductive failure are provided. The kits contain: (a) one or more reagents for testing a biological sample obtained from the subject for the presence of a variant in the IL-1 beta promoter region and/or a variant in the CD46 gene intron 1 region; and (b) instructions for using the one or more reagents to determine the presence of a variant in the IL-1 beta promoter region and/or the presence of a variant in the CD46 gene intron 1 region to determine whether the mammal has a predisposition to recurrent pregnancy loss. In certain preferred embodiments, the kits contain reagents to detect the presence of one or more of the following: a variant of the IL-1 beta gene at position-511; a variant of the IL-1 beta gene at position-31; and/or a variant in the CD46 gene intron 1 region. In certain embodiments, the reagents for testing the subject for the presence of the variant in the IL-1 beta promoter region include a pair of amplification primers for a polymerase chain reaction amplification of the promoter region defining the polymorphism. In certain embodiments, the reagents for testing the subject for the presence of variants in the CD46 gene intron 1 region include a pair of amplification primers for a polymerase chain reaction amplification of the intron 1 region defining the polymorphism.

According to a fourth embodiment, a method for treating a subject diagnosed as having recurrent pregnancy loss and suspected of having immunologic reproductive failure is provided. The method involves: (a) selecting a subject having a variant in the IL-1B beta promoter region and/or having a variant in the CD46 gene intron 1 region; and (b) administering to the subject an effective amount of an immunomodulating agent to prevent or reduce the occurrence of recurrent pregnancy loss. The immunomodulating agent is selected from the group consisting of an immunomodulating agent that downregulates a T_{H}-1 immune response and an immunomodulating agent that upregulates a T_{H}-2 response. Exemplary immunomodulating agents are glucocorticoids, cyclosporins, nifidipine, pentoxiphylline, progesterone and intravenous immunoglobin. Progesterone is a particularly preferred immunomodulating agent.

These and other aspects of the invention, as well as various advantages and utilities, will be more apparent with reference to the detailed description of the preferred embodiments.

### BRIEF DESCRIPTION OF THE DRAWINGS

This application, including the examples, may refer to tables, drawings, and, optionally, color representations; however, the tables, drawings, or other representations are not required for enablement of the inventions disclosed herein.

FIG. 1. (Example 1) Genotypes for IL-1b in fertile controls and women with RPL who had or did not have evidence of Thl immunity to trophoblast. The frequencies of homozygotes for variant IL1B-511C (C/C), homozygotes for IL1B-511T (T/T), and heterozygotes for the two variants (C/T) were compared among three groups [fertile controls, RPL Th1(−), and RPL Th1(+) group 1+2]. The P values that show statistically significant differences in frequencies of the genotype indicated.

FIG. 2. (Example 1) Correlation of trophoblast extract-induced PBMC IFN-g production with IL1B genotype in women with RPL. The study subjects were grouped by IL1B genotype as indicated: IL1B-511C carrier (C+) and noncarrier (C−) in panel a and C/C, C/T and T/T genotypes in panel b. The levels of IFN-g production (background subtracted) by PBMCs (stimulated with trophoblast extracts) from women with RPL were presented by box plots. Lines of the boxes delineate the 25th, 50th, and 75th percentile (from bottom to top). The top and bottom short lines depict the 90th and 10th percentile of the population, respectively. The notch defines the 95% confidence interval around the median (50th percentile). Groups that display nonoverlapping notches are statistically significantly different (P<0.05). Data were analyzed with the Mann-Whitney test (C+vs C−, respectively; P=0.04) in panel A and the Kruskal-Wallis one-way ANOVA (C/C vs T/T, respectively; P=0.028; C/C vs C/T or C/T vs T/T, respectively; P>0.05) in panel B.

FIG. 3. (Example 1) Differential effects of trophoblast and allogenic PBMC extracts on in vitro IFN-g production by PBMCs in women with RPL. PBMCs from 14 women with unexplained RPL were stimulated for 5 days with extracts (30 mg of protein/ml) from the trophoblast lineage cell line Jeg-3 (Troph Ext) or allogenic PBMC extracts (PBMC Ext). Red cell membrane (Red Cell) was used as a negative control, and irradiated allogenic PBMCs (PBMCs) and PHA were used for comparison. Supernatants were then tested for IFN-g with an ELISA kit. The IFN-g production levels are subtracted with background (IFN-g production by PBMCs incubated with medium alone) are presented as spot plots.

FIG. 4. (Example 1) Results of Example 1 are shown.

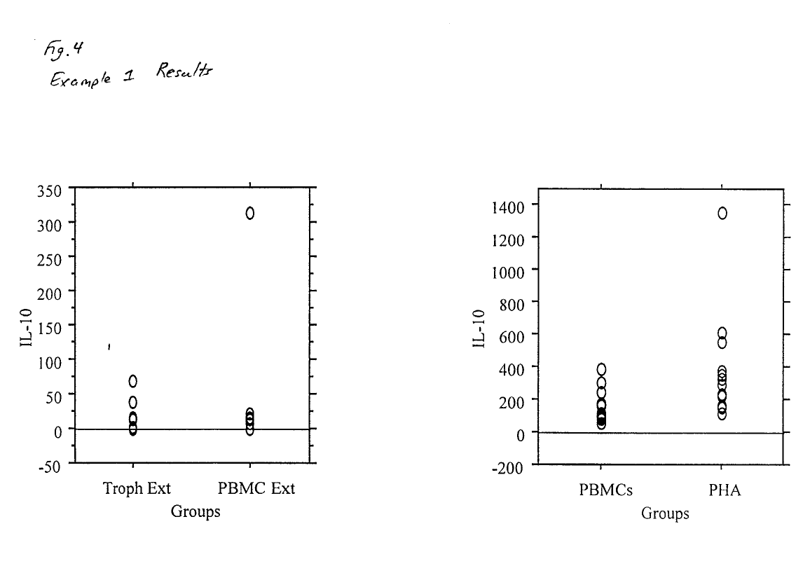

FIG. 5 (Example 3) illustrates the correlation of trophoblast extract-induced PBMC IFN-γ production with IL1B-511 genotype in women with a history of RPL. PBMCs from study subjects were cultured in the presence or absence of a protein extract (30 μg/ml) from trophoblast lineage cell line Jeg-3 for 5 days as described.⁵ Culture supernatants were harvested and tested for IFN-γ concentration using a ELISA kit (Endogen, Cambridge, Mass.). The study subjects were grouped by IL1B genotype as indicated: IL1B-511C carrier (C⁺) and noncarrier (C⁻) in panel a and C/C, C/T and T/T genotypes in panel b. The levels of IFN-γ production (background subtracted) were presented by box plots. Lines of the boxes delineate the 25^{th}, 50^{th}, and 75^{th} percentile (from bottom to top). The top and bottom short lines depict the 90^{th} and 10^{th} percentile of the population, respectively. The notch defines the 95% confidence interval around the median (50^{th} percentile). Data were analyzed with the Mann-Whitney rank in panel A and with the Kruskal-Wallis rank test in panel B.

### INTRODUCTION TO SEQUENCE LISTING

The Example discloses variants of the IL-1 beta gene promoter region and of the CD46 gene intron 1, and identifies the locations of these variants. The sequence for the IL-1 promoter region previously has been described (see, e.g., GenBank Accession No. X04500; Clark, B. D., et al., Nucleic Acids Res. 14(20), 7897-7914 (1986); Guasch, J. F., et al., Cytokine 8(8), 598-602 (1996); and di Giovine, F.S., et al., Human Molecular Genetics 1(6), 450 (1993); The sequence for the CD46 gene intron 1 also previously has been described (see, e.g., Bora, N. S., et al., J. Immunol. 146(8), 2821-2825 (1991)). These sequences are described in more detail in the Examples and the accompanying Sequence Listing.

### DETAILED DESCRIPTION OF THE INVENTION

The invention is based, in part, on the discovery of an association between the variants of the Interleukin-1 beta gene promoter and/or variants of the CD46 gene intron 1 and unexplained recurrent pregnancy loss. Although not wishing to be bound to any particular theory or mechanism, Applicants believe that such unexplained recurrent pregnancy loss is potentially mediated by T-helper type 1 immunity to trophoblast. Accordingly, the compositions and methods of the present invention are directed to compositions containing the isolated variants, reagents for the detection of such variants, and functional equivalents of the foregoing, and the use of such compositions for diagnosing and/or treating RPL and for research applications. Thus, these isolated genetic markers for RPL and/or the reagents for their detection can be incorporated into assay screening kits for use in, for example, hybridization assays, to identify women having a propensity to RPL who carry one or more of these genetic markers. The identification of the genetic marker(s) for RPL, therefore, permits a more accurate prediction and diagnosis of this condition than has heretofore been possible. Thus, the invention encompasses compositions, as well as diagnostic and therapeutic methods that are useful for diagnosing and/or treating RPL.

Applicants have discovered an association between the variants at positions-511 and −31 of the Interleukin-1 beta gene promoter region and/or between variants in the CD46 gene intron 1 and unexplained recurrent pregnancy loss potentially mediated by T-helper type 1 immunity (e.g., to trophoblast). This discovery has utilities in the diagnosis and treatment of conditions associated with women's health in general and, in particular, pregnancy, genetics and immunology. More specifically, this invention belongs to a conceptual observation regarding the discovery of a novel relationship between two variants of the human Interleukin IL-1 gene (IL1B) promoter region and T-helper type-1 (Th1) immunity and/or variants in the CD46 gene intron 1 in unexplained recurrent pregnancy loss (URPL). The two IL1B variants are characterized by a base C at position −511 (IL1B-511C) and a base T at position −31 (IL1B-31T) from the transcriptional start site of IL1B gene. The CD46 gene intron 1 variants are characterized by a change in the HindIII site in this intron. Compared with fertile controls, the frequencies of these variants, alone or together, are significantly increased in women with unexplained recurrent pregnancy loss associated with biased Th1 immunity to trophoblast as evidenced by high IFN-γ production by their peripheral blood mononuclear cells following exposure to extract(s) derived from a trophoblast cell line, JEG-3. Because Th1 cytokines, such as IFN and TNF may disrupt a number of normal reproductive process, a biased Th1 cytokine response could contribute to pregnancy failure. On the other hand, IL-1 has effects on regulation of either Thl or Th2 cytokine response, and the two variants of the IL1B promoter region have been reported to potentially affect IL-1 production. Therefore, our observations suggest that these two promoter variants of IL1B gene and the CD46 gene intron 1 are involved in the etiology of recurrent pregnancy loss in a subgroup of women with evidence of a Th1 biased cytokine response. Thus, we believe these IL1B promoter variants and CD46 gene intron 1 variants are useful as genetic markers for the diagnosis and management of this disorder. Therefore, identification of these IL1B promoter variants and/or identification of CD46 gene intron 1 variants represents a novel method of determining a woman's propensity of having reproductive failure attributed to Th1 cytokines.

In view of the foregoing, the invention provides for the use of IL-1 beta promoter variants and CD46 variants as genetic markers for human reproductive failure. Accordingly, Applicants disclose herewith for the first time, evidence that this genetic polymorphism is associated with human reproductive difficulty, and propose that determining these variants may be useful in the diagnosis and management of recurrent pregnancy loss. Although not wishing to be bound to any particular theory or mechanism, we believe that such polymorphisms of IL-1 beta promoter and polymorphisms of the CD46 gene intron 1 region facilitate a Th1 cytokine response potentially culminating in reproductive failure. Thus, the invention has utility for health care providers interested in the diagnosis and treatment of women with reproductive failure (infertility, recurrent pregnancy loss). Such diagnostic methods will allow the identification of women with a propensity to make a Th1 cytokine response as a cause of their reproductive difficulty, e.g., by determining whether they have a polymorphism for the IL-1 beta promoter region and/or a polymorphism for the CD46 gene intron 1 region. Identification of these variants, thus allows physicians and other health care providers to define women more likely to benefit from immunomodulating therapy.

According to first aspect of the invention, a method for evaluating a risk of recurrent pregnancy loss in a subject suspected of having immunologic reproductive failure is provided. The method involves: (a) testing a biological sample obtained from the subject for the presence of: (1) a variant in the IL-1 beta promoter region and (2) a variant in the CD46 gene intron 1 region, wherein the presence of the variant in the IL-1 B beta promoter region and the variant in the CD46 gene intron 1 region is indicative of an elevated risk of developing recurrent pregnancy loss. In preferred embodiments, the variant in the IL-1 beta promoter region is a polymorphism of the IL-1 beta gene at position-511 and/or a polymorphism of the IL-1 beta gene at position-31 and the variant in the CD46 gene intron 1 region is a polymorphism in the CD46 gene intron 1 region in the HindIII restriction site. Exemplary biological samples include cell (e.g., leukocyte)-containing samples, e.g., peripheral blood and serum, peritoneal fluid, endometrial tissue, vaginal secretions and saliva. Methods for obtaining the above-described samples from a patient are known to one of ordinary skill in the art. The cell-containing sample can be used in the methods of the invention with or without prior culturing. Preferably, the cell-containing sample is isolated from the mammal, and the nucleic acid components are processed in accordance with standard methods to determine the presence of a variant of the IL-1 beta promoter and/or the presence of a variant of the CD46 gene intron 1 region.

According to second aspect of the invention, a method for evaluating a risk of recurrent pregnancy loss in a subject suspected of having immunologic reproductive failure is provided which involves: (a) testing a biological sample obtained from the subject for the presence of: (1) a variant in the IL-1 beta promoter region or for (2) a variant in the CD46 gene intron 1 region, wherein the presence of the variant in the IL-1B beta promoter region and/or the variant in the CD46 gene intron 1 region is indicative of an elevated risk of developing recurrent pregnancy loss. The preferred embodiments detect the variants disclosed in reference to the first aspect of the invention, namely, a polymorphism of the IL-1 beta gene at position-511, a polymorphism of the IL-1 beta gene at position-31, or a polymorphism in the CD46 gene intron 1 region in the HindIII restriction site.

According to a third aspect of the invention, kits useful for evaluating the risk of recurrent pregnancy loss in a subject suspected of having immunologic reproductive failure are provided. The kits contain: (a) one or more reagents for testing a biological sample obtained from the subject for the presence of a variant in the IL-1 beta promoter region and/or a variant in the CD46 gene intron 1 region; and (b) instructions for using the one or more reagents to determine the presence of a variant in the IL-1 beta promoter region and/or the presence of a variant in the CD46 gene intron 1 region to determine whether the subject has a predisposition to immunologic reproductive failure. In certain preferred embodiments, the kits detect the presence of one or more of the following: a variant of the IL-1 beta gene at position-511; a variant of the IL-1 beta gene at position-31; and/or a variant in the CD46 gene intron 1 region. In certain embodiments, the reagents for testing the sample for the presence of the variant in the IL-1 beta promoter region include a pair of amplification primers for polymerase chain reaction amplification of the promoter region defining the polymorphism. In certain embodiments, the reagents for testing the subject for the presence of variants in the CD46 gene intron 1 region include a pair of amplification primers for polymerase chain reaction amplification of the intron 1 region defining the polymorphism.

In particular, the invention enables the diagnosis of immunologic recurrent pregnancy loss associated with Thl-type immunity (e.g., to trophoblast). This technology involves the use of restriction fragment length polymorphism (RFLP) for typing genetic variants of IL-1B-511, IL-1 B-31, and CD46 gene intron 1. Thus, the invention advantageously provides for the first time a genetic diagnosis of recurrent pregnancy loss potentially due to Th1-type immunity. The particular methods for performing the genetic testing are those available to one of skill in the art. For example, the RFLP method for typing genetic variants has been reported in numerous United States patents for diagnosing conditions associated with a genetic polymorphism. (See, e.g., U.S. Pat. No. 6,030,778 entitled Diagnostic Assays and Kits for Body Mass Disorders Associated with a Polymorphism in an Intron Sequence of the SR-BI Gene; U.S. Pat. No. 6,027,913 entitled Nucleic Acid Amplification with Direct Sequencing; U.S. Pat. No. 5,994,080 entitled Method of Diagnosing an Increased Risk of Thrombus Associated Disease by Detecting a Certain t-PA Polymorphism; U.S. Pat. No. 5,922,575 entitled Mutations in the katg Gene Useful for Detection of M. tuberculosis; U.S. Pat. No. 5,912,127 entitled Method and Kit for Evaluating Risk of Ovarian Cancer in Carriers of a BRCA1 Mutation; U.S. Pat. No. 5,876,927 entitled Nucleic Acid Diagnostic Assay for Charcot-Marie-Tooth Disease Type 1B; and U.S. Pat. No. 5,863,772 entitled Method of Individual Discrimination by Polymerase Chain Reaction using M1 Primer.)

In addition, binding molecules (e.g., nucleic acids) complementary to the sequences containing the specific nucleotide changes described herein, can be used to identify such polymorphisms in vivo or in vitro. Accordingly, such binding molecules are useful for diagnostic applications (e.g., wherein the binding molecule hybridizes under stringent conditions to the portion of the promoter region or intron region containing the variant nucleotide(s)) and contains a marker which can be directly detected or indirectly detected (e.g., via a linker molecule such as biotinylated binding molecule detected by Avidin associated detectable marker-radiolabel, enzyme, and so forth). Such binding molecules are useful for diagnostic applications as well as for therapy.

It should be understood that kits which include reagents that are useful for detecting the polymorphism can be assembled which provide convenient access and use in clinical settings. For example, a kit can include a container which holds one or more amplification primers, a container which holds enzymes used for amplification, a container which holds washing solution(s), a container which holds detection reagents, and a sample well. It is also contemplated that a kit can include a container having one or more labeled or unlabeled probes capable of selectively hybridizing to the specific variants described herein of the IL-1 beta gene promoter or to the specific variants described herein of the CD46 gene intron 1, a container having one or more labeled or unlabeled probes capable of hybridizing to this specific region and, if the probe is unlabeled, a container having a labeled specific binding partner of the probe or to a recognition site on the probe, e.g., a biotinylated probe, a container which holds washing solution(s), a container which holds detection reagents and a sample well. Alternatively, a kit may contain a single probe which is capable of hybridizing selectively to the particular region of the IL-1 beta promoter region or to the particular region of the CD46 gene intron 1 containing the polymorphisms disclosed herein along with other suitable components such as washing solution(s) and the like.

Examples of detection reagents include radiolabeled probes, enzymatic labeled probes (horseradish peroxidase, alkaline phosphatase), and affinity labeled probes (Biotin, Avidin, or Streptavidin). For antibodies, examples of detecting reagents include, but are not limited to, labeled secondary antibodies, or if the primary antibody is labeled, the chromophoric, enzymatic, or antibody binding reagents which are capable of reacting with the labeled antibody. The antibodies, primers, and nucleic acid probes described herein can readily be incorporated into one of the established kit formats which are well known in the art.

It is also contemplated that transgenic animals may be generated which contain the IL-1 beta gene promoter region polymorphisms and/or the CD46 gene intron 1 polymorphisms disclosed herein, which animals are useful in studying the effect of the polymorphism on immunological response and, more importantly, to identify and screen reagents which may be useful for inhibiting or otherwise minimizing recurrent pregnancy loss. Methods of creating transgenic animals are well known in the art. For example, U.S. Pat. No. 4,873,191, incorporated herein by reference, describes genetic transformation of zygotes. Following such procedures, the IL-1 beta gene and/or the CD46 gene containing the polymorphism disclosed herein is microinjected into the nucleus of a zygote which is then allowed to undergo differentiation and development into a mature organism. Transgenic animals, such as mice or pigs, will have somatic and germ line cells containing the IL-1 beta gene and/or CD46 gene variants. Such animals are useful as in vivo models for recurrent pregnancy loss and allow for the further development and testing of treatment modalities.

According to a preferred aspect of the invention, isolated nucleic acids which hybridize under stringent conditions to the region of the IL-1 beta promoter or the region of the CD46 gene intron 1 containing the polymorphisms disclosed herein are provided. These isolated nucleic acids bind selectively to the variants of the IL-1 beta gene promoter region or to variants of the CD46 gene intron 1 disclosed herein. The isolated nucleic acids may be further contained in kits, as described above for the diagnosis of a propensity to recurrent pregnancy loss.

According to yet another aspect of the invention, a method for evaluating risk of a female subject (a mammal such as a human, horse, dog, sheep, cow, cat, rat, a mouse and so forth) for recurrent pregnancy loss is provided. The method involves testing a biological sample obtained from the subject for the presence of the variant(s) of the IL-1 beta gene promoter region and/or CD46 gene intron 1 region disclosed herein, wherein the presence of one or more of the variants is indicative of an elevated risk of developing recurrent pregnancy loss. The preferred method for evaluating the risk of recurrent pregnancy loss involves testing the biological sample for the presence of both an IL-1 beta promoter variant and a CD46 gene intron 1 variant. Preferably, the method involves amplifying the IL-1 beta gene promoter region and the CD46 gene intron 1 region to form amplification products and determining whether the products contain the variants disclosed herein. Exemplary procedures for amplification and general methods for testing a subject for the presence of a polymorphism are described in U.S. Pat. No. 5,912,127, issued to Marod et al., “Method and Kit for Evaluating Risk of Ovarian Cancer in Carriers of a BRCA1 Mutation”. In the preferred embodiment, testing comprises using restriction fragment length polymorphism (RFLP) and, optionally, determining the frequency of the variant. An exemplary kit for evaluating risk of recurrent pregnancy loss in a subject comprises in packaged combination: (a) one or more reagents for testing the subject for the presence of the one or more variants of the IL-1 beta gene promoter region and/or the CD46 gene intron 1 disclosed herein; and, optionally (b) one or more pairs of amplification primers for polymerase chain reaction amplification of these polymorphic regions.

According to a fourth embodiment, a method for treating a subject diagnosed as having recurrent pregnancy loss and suspected of having immunologic reproductive failure is provided. The method involves: (a) selecting a subject having a variant in the IL-1B beta promoter region and/or having a variant in the CD46 gene intron 1 region; and (b) administering to the subject an effective amount of an immunomodulating agent to prevent or reduce the occurrence of recurrent pregnancy loss. The immunomodulating agent is selected from the group consisting of an immunomodulating agent that downregulates a T_{H}-1 immune response and an immunomodulating agent that upregulates a T_{H}-2 response. Exemplary immunomodulating agents are glucocorticoids, cyclosporins, nifidipine, pentoxiphylline, progesterone and intravenous immunoglobin. Progesterone is a particularly preferred immunomodulating agent.

In contrast to the methods disclosed in the prior art for the detection of subjects with a predisposition to immunologic reproductive failure, the methods disclosed herein permit the detection of a select population of subjects who will benefit from an immunomodulating treatment regimen. Accordingly, the instant invention provides a method for treating immunologic reproductive failure, which includes administering one or more immunomodulating agents to this population of subjects.

As used herein, an “immunomodulating agent” refers to an agent capable of modulating a cellular immune response and includes agents which directly or indirectly modulate the effective cytokine concentration(s) in vivo. The immunomodulating agent can be a nonspecific immunomodulating agent (i.e., not targeted to modulating an immune response to a particular target antigen) that downregulates a T_{H}-1 immune response or that upregulates a T_{H}-2 immune response. Exemplary nonspecific immunomodulating agents include glucocorticoids, cyclosporins, nifidipine, pentoxiphylline and progesterone. Alternatively, the immunomodulating agent can be a specific immunomodulating agent that modulates the cellular immune response to a specific reproductive antigen. According to a particularly preferred embodiment, the specific immunomodulating agent is a vaccine including a reproductive antigen contained in an adjuvant. An adjuvant is selected that downregulates a T_{H}-1 type immune response or that upregulates a T_{H}-2 type immune response to the reproductive antigen in vivo. Oral vaccines for modulating a cellular immune response to a specific antigen have been described (see, e.g., PNAS, USA 91:437-438 (1994); Immunology Today 12:383-385 (1991); Cellular Immunology 131:302 (1990); PNAS, USA 89:421-425 (1992); Science 259:1321-1324 (1993); and Science 261:1727-1730 (1993), the contents of which references are incorporated herein by references). An exemplary cellular vaccine of the invention is an oral vaccine prepared by placing the Jeg-3 antigen in adjuvants such as those described in the above-identified references. Additional cellular vaccines containing reproductive antigens such as trophoblast antigens are described in International Application No. PCT/US94/05692, filed MAY 20, 1994.

Adjuvants which regulate a T_{H}-1 type response and/or a T_{H}-2 type response can be selected by determining the T_{H}-1 and/or T_{H}-2 cytokine profile following immunization of, for example, an animal with a vaccine containing a test antigen (e.g., BSA, reproductive antigen) contained in the test adjuvant. Exemplary adjuvants that upregulate a T_{H}-2 type response (and thereby downregulate a T_{H}-1 response) include alum and squalene in oil. Additional adjuvants which can be screened for their ability to upregulate (or downregulate) a T_{H}-1 type response and/or a T_{H}-2 type response include the ISCOMS (Morein, B., et al., Nature (Lond.). 308:457-459 (1984)), cholera toxin adjuvants (Quiding, M., et al., J. Clin. Invest. 88:143-148 (1991)) and complete Freund's adjuvant. The ISCOMS are prepared by removing detergent in a controlled fashion from a mixture of cholesterol, protein, phospholipid, detergent and Quil-A (e.g., by dialysis and centrifugation on a Quil-A-containing sucrose gradient). ISCOM formation is confirmed by negative contrast electron microscopy and by their distinctive sedimentation constant (19S) in a sucrose gradient. Quil-A is a saponin extracted from the bark of the tree Quillaja saponaria. Purification of these saponins and their use as adjuvants is described in U.S. Pat. No. 5,057,540, issued to Kensil et al., the contents of which patent are incorporated herein by reference.

As used herein, “effective cytokine concentration” refers to the concentration of cytokine that is available for binding to cytokine receptors, i.e., the concentration of cytokine that is capable of triggering a cellular immune response. Thus, immunomodulating agents embrace agents which function by (1) reducing T_{H}-1 cytokine release from leukocytes; (2) reducing the concentration of receptors capable of binding to the T_{H}-1 cytokines; (3) binding directly to T_{H}-1 cytokines, thereby preventing cytokine binding to receptors; (4) competing with the T_{H}-1 cytokines for binding to cytokine receptors; as well as (5) agents which modulate the concentration of any of the above (e.g., T_{H}-² cytokines). Accordingly, immunomodulating agents include agents which are known in the art for their ability to suppress an immune response (e.g., progesterone), as well as TGF-beta and antibodies to the embryotoxic cytokines and/or antibodies to the embryotoxic cytokine receptors (e.g., antibodies to gamma-interferon, tumor necrosis factor-alpha, interleukin-2 and interleukin-6 or antibodies to cytokine producing cells such as CD-3 and CD56 cells or to their receptors). In a preferred embodiment, the immunomodulating agent is capable of modulating the cellular immune response in a localized area, i.e., the area in fluid or tissue communication with fetal cells, as distinguished from a humoral immune response.

The immunomodulating agents are administered in therapeutically effect amounts. A therapeutically effective amount is that amount which is sufficient to reduce or prevent the occurrence of reproductive failure in the treated subject. The effective amount of agent will depend upon the clinical condition of the subject being treated. A therapeutically effective amount can be determined in a number of ways using medical techniques customary to one of ordinary skill in the art. For example, different amounts of immunomodulating agent can be administered to selected subjects having one or more of the polymorphisms disclosed herein. The therapeutically effective dose of immunomodulating agent is selected which reduces the occurrence of reproductive failure in the selected subject.

The selection of a therapeutically effective dose of the immunomodulating agent to prevent immunologic reproductive failure is made in accordance with standard procedures known to one of ordinary skill in the art, taking into consideration the patient's clinical condition. Such amounts will depend, of course, on the particular condition being treated, the severity of the condition, and individual patient parameters including age, physical condition, size, weight and concurrent treatment. These factors are well known to those of ordinary skill in the art and can be addressed with no more than routine experimentation. It is preferred generally that a maximum dose be used, that is, the highest safe does according to sound medical judgment. It will be understood by those of ordinary skill in the art, however, that a patient may insist upon a lower dose or tolerable dose for medical reasons, psychological reasons or for virtually any other reasons.

Administration of the immunomodulating agent is performed in accordance with methods known to one of ordinary skill in the art. Accordingly, a variety of administration routes are available. The particular mode selected will depend, of course, upon the particular drug selected, the particular condition being treated and the dosage required for therapeutic efficacy. The methods of this invention, generally speaking, may be practiced using any mode of administration that is medically acceptable, meaning any mode that produces therapeutic levels of the agents of the invention without causing clinically unacceptable adverse effects. Such modes of administration include oral, rectal, vaginal, topical, transdermal or parenteral (e.g. subcutaneous, intramuscular and intravenous) routes. Formulations for oral administration include discrete units such as capsules, tablets, suppositories, patches, lozenges and the like. For an example of a vaginal suppository containing an immunomodulating agent (progesterone), see U.S. Pat. No. 5,084,277, the contents of which patent are incorporated herein by reference.

The compositions may conveniently be presented in unit dosage form, including oral vaccine form, and may be prepared by any of the methods well known in the art of pharmacy. Such methods include the step of bringing the active agents into association with a carrier which constitutes one or more accessory ingredients. In general, the compositions are prepared by uniformly and intimately bringing the agents into association with a liquid carrier, a finely divided solid.

The invention will be more fully understood by reference to the following examples. These examples, however, are merely intended to illustrate the embodiment of the invention and are not to be construed to limit the scope of the invention. It is also to be understood that the referenced figures and tables are illustrative only and are not essential to the enablement of the invention as disclosed herein.

### EXAMPLES

### Example 1 Description

Objectives: Biased Th1 immunity to trophoblast and defective Th2 cytokine production by decidual T cells have been associated with RPL. The underlying genetic mechanisms remain to be elucidated. Our preliminary studies did not show association of polymorphisms of the IFNG gene and the TNFA promoter region with Th1 immunity to trophoblast. Because IL-1 may influence either Th1 or Th2 immune response, in the present study, we investigated whether polymorphisms of the ILI B and IL1 RN genes influence Th1 immunity to trophoblast in women with RPL.

Design: A case control study was performed in Caucasian women with a history of three or more pregnancy loss from the Center for Reproductive Medicine of our hospital. Patients were grouped into Th1 (+) and Th1(−) subgroups depending upon IFN-γ production by their trophoblast-stimulated PBMC in vitro as evidence of Thl immunity to trophoblast. Caucasian women with a history of two or more successful pregnancies and no pregnancy loss were recruited as fertile controls.

Materials and Methods: Diallelic polymorphisms of the IL1B gene at positions-511 and +3953 were analyzed using PCR-restriction fragment length polymorphism (RFLP) methods and tandem repeat polymorphism of the IL1RN gene was analyzed by PCR and gel electrophoresis. IFN-γ production by trophoblast-stimulated PBMC was determined by ELISA. Data were statistically analyzed by Fisher's Exact Text or by Kruskal-Wallis one way ANOVA and Mann-Whitney rank sum Test.

Results: The frequency ofIL1B-511 allele 1 was significantly increased in the Th1(+) RPL group (allele n=96) compared to fertile controls (allele n=122)(76% vs. 51.6%; P=0.0002), compared to the Th1(−) RPL group (allele n=92)(76% vs. 60.8%; P+0.028). An increased frequency of this allele was confirmed in a second Th1(+) RPL group (allele n=84), compared to fertile controls (67.8% vs. 51.6%; P=0.022). These changes correlated with an increased frequency of the homozygotes for IL1B-511 allele 1 in the two Th1(+) RPL groups compared to fertile controls (P=0.0023). No significant difference in the allelic frequencies of IL1B+3953 and ILRN gene polymorphisms was observed between all the study groups. The high level IFN-γ production by trophoblast-stimulated PBMC was associated with IL1B-511 allele 1 in women with RPL (P<0.05).

Conclusions: These results suggest that polymorphism of the IL1B promoter region may influence Th1 immunity to trophoblast in women with RPL and IL-1 may be involved in the regulation of Th1/Th2 cytokine production in human reproduction.

T-helper 1-type immunity to trophoblast in women with recurrent pregnancy loss is associated with polymorphisms of the IL1B promoter region.

Recurrent pregnancy loss (RPL) is a common disorder during early gestation, the cause of which remains unexplained in approximately 50% of cases^{1,2}. Recent research suggests that T-helper 2 (Th2)-type cytokine production at the fetal-maternal interface is necessary for successful gestation³ and that a shift from Th2 towards Th1-type cytokine production⁴⁻⁶ and defective Th2 cytokine production by T cells in the decidua⁷ are associated with RPL. Because Th1 cytokines, e.g., interferon-g (IFN-g) and tumor necrosis factor (TNF), may disrupt a number of normal reproductive processes⁸⁻¹⁰, a biased Th1 cytokine response could contribute to reproductive failure¹¹. The potential molecular genetic mechanisms underlying such Thl-type bias are poorly understood. Polymorphisms of the genes for IFN-g, TNFa, and interleukin (IL)-1b have been shown to influence cytokine production and thus contribute to susceptibility of certain diseases¹²⁻¹⁵. We investigated polymorphisms of the genes for IFN-g (IFNG) and for TNFa (TNFA) in women with RPL and found no association between polymorphism of these genes and Th1 immunity to trophoblast. However, additional investigation of polymorphisms in the IL-1b gene (IL1B) showed a significant increase in the frequency of a variant at position 511 of the IL1B promoter region (IL1B-511C) in women with RPL who had evidence of Th1-type immunity to a protein extract derived from the trophoblast cell line Jeg-3 as previously described ^{4,16} High IFN-g production by peripheral blood mononuclear cells (PBMCs) stimulated with trophoblast extracts in women with RPL correlated with IL1B-511C. These results suggest that polymorphisms of the IL1B promoter region influence Th1 immunity to trophoblast in women with RPL. We propose that IL-1 is involved in the regulation of Th1/Th2 cytokine production in human reproduction.

A disproportionately high production of Th1 type cytokine IFN-g and low production of Th2 cytokine IL-10 by PBMCs stimulated with protein extracts derived from the trophoblast lineage cell line Jeg-3 as well as with autologous trophoblasts has been demonstrated in some women with RPL^{4,5}. Such high IFN-g production in vitro is likely to reflect a Th1-type bias in cytokine response in vivo. Thus, based on IFN-g production by PBMCs following in vitro exposure to trophoblast extracts, it is possible to subgroup women with RPL for studies on genetic contributions to immunity in RPL. In the present study, we investigated whether polymorphisms in the genes for Th1 cytokines IFN-g and TNF-a and for IL-1b were associated with RPL potentially mediated by Th1 immunity to trophoblast. To this end, we initially studied 94 Caucasian women with a history of unexplained RPL. Forty-eight had produced high levels of IFN-g in response to trophoblast extracts (mean level 213.28±34.07 pg/ml) and were designated the RPL Th1(+) subgroup, while 46 had not produced IFN-g and were designated the RPL Th1 (−) subgroup. Additional 61 Caucasian women who had experienced at least two successful pregnancies with no history of pregnancy loss were recruited as fertile controls. A second RPL Th1(+) group (n=42) was subsequently studied for verification. In an initial study, typing for CA-repeat polymorphism in the first intron of IFNG revealed similar distributions of IFNG alleles in the two RPL subgroups and fertile controls (Table 1). Subsequent investigation of polymorphisms in the TNFA promoter region showed no statistical difference in frequencies of the 5 variants among all the RPL subgroups and the fertile controls (Table 1).

We next investigated polymorphisms of the IL1B gene and the IL-1 receptor antagonist (IL-1Ra) gene (IL1RN) in women with RPL, because cytokines in the IL-1 system [IL-1a, IL-1b and IL-1Ra] are produced at the fetal-maternal interface¹⁷ and may be involved in the regulation of Th1/Th2 cytokine production at this site as they can influence natural killer (NK) and T cell IFN-g production^{18, 19} and function as a co-stimulator for Th2 cell proliferation ²⁰⁻²² Moreover, IL-1 has been implicated in implantation and trophoblast invasion ^{23,24}.

Biallelic base substitution (C to T) polymorphisms have been described at positions 511 and +3953 bases away from the transcriptional start site of the IL1B gene ^{25,26}. Polymorphisms of the IL1B and IL1RN genes may influence the course and severity of certain inflammatory diseases ^{14, 15.} Because polymorphisms of the IL1B and IL1RN genes influence IL-1b and IL-1Ra production ^{15,27}, we investigated whether they were associated with Th1-type immunity in women with RPL. Experiments using restriction fragment length polymorphism (RFLP) analysis revealed that the frequency of the variant IL1B-511 C of the IL1B promoter region was significantly higher in women with RPL who had evidence of Th1 immunity to trophoblast than in women with RPL who did not have such evidence (76% vs 60.8%, respectively; P=0.028; odds ratio (OR)=2.04; 95% confidence intervals (CI): 1.088 to 3.826; Table 2). The frequency of variant IL1B-511C inthe RPL Th1(+) group was also significantly higher than that previously published ²⁵ for other Caucasian populations (76% vs 59%, respectively; P=0.021). These observations led us to hypothesize that polymorphism of the IL1B promoter region influences Th1-type immunity to trophoblast in women with RPL. Further experiments comparing women with RPL to fertile controls revealed a significantly increased frequency of IL1B-511C in the RPL Th1(+) group (76% vs 51.6%, respectively; P=0.0002; OR=2.972; 95% CI: 1.651 to 5.352; Table 2). There was no significant difference in the distributions of the alleles of IL1B at position +3953 and of IL1RN among our study groups.

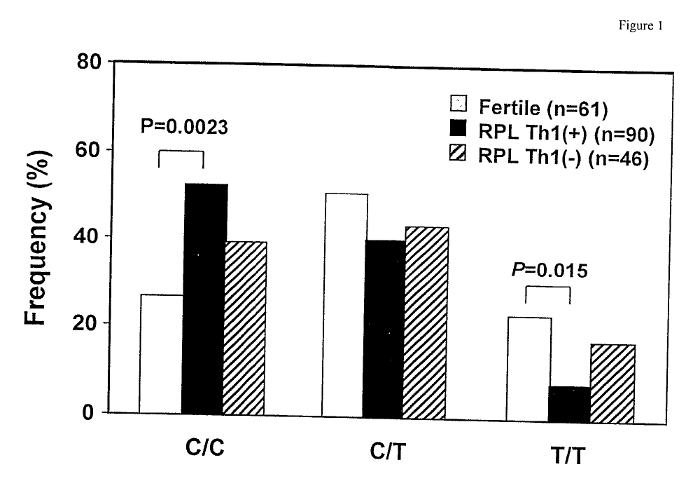

To confirm our results, we subsequently tested an additional group of women with RPL who had evidence of Thl immunity to trophoblast (n=42) and again found an increased frequency of IL1B-511C, compared with fertile controls (67.8% vs 51.6%, respectively; P=0.022; OR=1.977; 95% CI: 1.107 to 3.530; Table 2). The increased frequency of this variant in the two RPL Th1(+) groups correlated with an increased frequency of the homozygotes for IL1B-511C (52.2% vs 26.2%, respectively; P=0.0023; OR=3.074; 95% CI 1.519 to 6.220), compared with fertile controls, and with a decreased frequency of the homozygotes for variants IL1B-511T (7.8% vs 22.9%, respectively; P=0.015; OR=0.283; 95% CI: 0.067 to 0.7510) in women with RPL who had evidence of Th1-type immunity to trophoblast (FIG. 1). However, there was no significant difference in the frequency of either IL1B-511C or homozygotes for this allele between the women with RPL who did not have evidence of Th1 immunity to trophoblast and the fertile controls. These results suggest that the IL1B-511C is associated with RPL in women with evidence of Th1-type immunity to trophoblast.

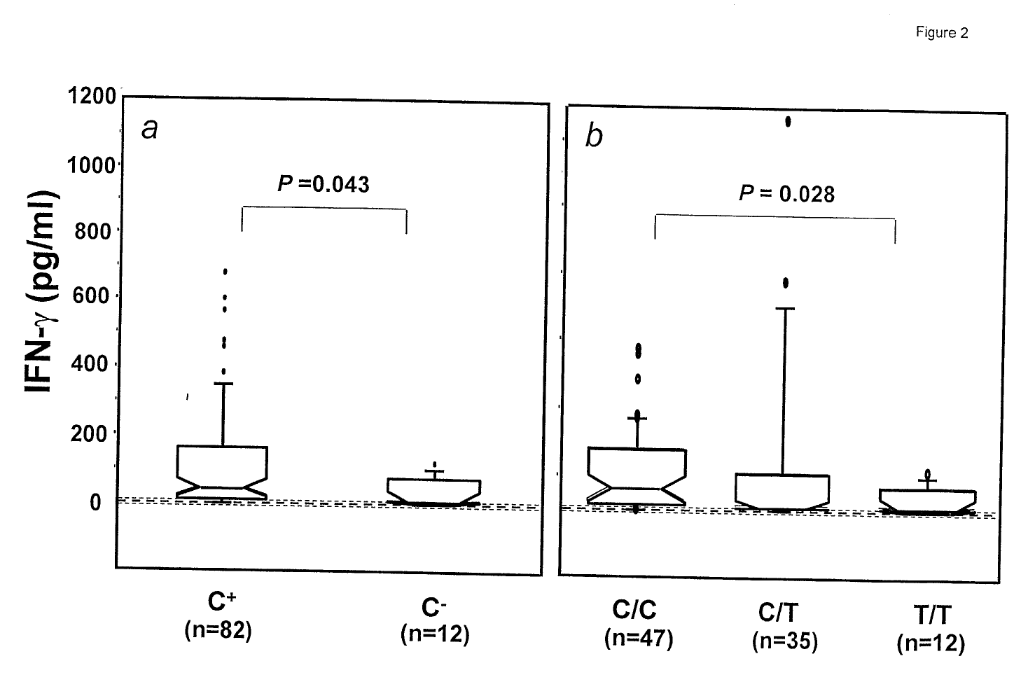

We next analyzed the relationship between variants at IL1B-511 and trophoblast-induced IFN-g production by PBMCs in women with RPL. As shown in FIG. 2a, high production of IFN-g occurred more frequently in IL1B-511C carriers (C+) than in IL1B-511 C noncarriers (C−) with RPL (median level 42.2 pg/ml vs 1.1 pg/ml, respectively; P=0.043; FIG. 2a). The ILI B-511C/C genotype was associated with high IFN-g production in response to trophoblast, compared with the IL1B-511T/T genotype in women with RPL (median level 63.8 pg/ml vs 1.1 pg/ml, respectively; P=0.028; FIG. 2b). These results are consistent with the hypothesis that polymorphism of the IL1B promoter region influences Th1 immunity to trophoblast in women with RPL.

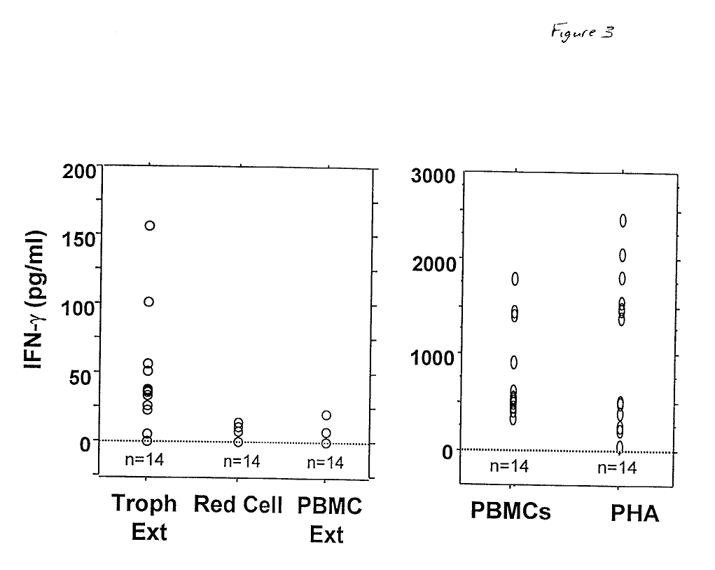

To determine whether IFN-g production by PBMCs stimulated with trophoblast extracts reflected a specific response to trophoblast-associated factors or a non-specific Th1-type cytokine response, we compared the induction of PBMC IFN-g production in vitro following co-culture with extracts derived from pooled allogenic PBMCs and red cell membrane with extracts from the trophoblast lineage cell line Jeg-3 in 14 women with RPL. As shown in FIG. 3, IFN-g was induced by trophoblast protein extracts in 10 out of 14 subjects (median 34.7 pg/ml range from 0 to 156 pg/ml) whereas, IFN-g was induced in only one study subjects in response to PBMC extracts (median 0 pg/ml, range from 0-20.2 pg/ml) and red cell membrane (median 0 pg/ml, range from 0-13.6 pg/ml) at a very low levels. These differences were statistically significant (P<0.001). In contrast, high levels of IFN-g were induced by irradiated intact allogenic PBMCs or by mitogen PHA. However, there was no correlation between the levels of IFN-g production induced by trophoblast extracts compared with that induced by allogenic PBMCs or by PHA (data not shown). These results suggest that the PBMC IFN-g response to trophoblast extracts in women with RPL is to trophoblast-associated factors rather than to soluble allogenic antigens, such as HLA-C antigens expressed by trophoblast cells.

Another locus at position 31 of the IL1B gene (IL1B-3 1) has been described to be in almost complete linkage disequlilibrium with IL1B-511²⁸. We found a similar distribution of IL1B-511C and IL1B-31T in our study and control groups (data not shown). Thus, variant IL1B-31T like IL1B-511C is also associated with IFN-g response to trophoblast in women with unexplained RPL. This variant, which involves the TATA box of the IL1B promoter, thus may be functionally important. However, no functional data currently exists addressing its potential on IL-1b production. Recent studies revealed that IL1B-511C was associated with decreased levels of IL-1b mRNA and protein produced by monocytes, compared with IL1B-511T in normal individuals^{15,27}. An increased frequency of homozygotes for IL1B-511C found in our present study in a subgroup of women with RPL is consistent with the phenotype of low IL-1b production for this allele. We cannot exclude the possibility that other gene(s) in linkage disequilibrium with IL1B-511C and IL1B-31T also play a role. (See Example 2).

To our knowledge, this is the first study demonstrating that polymorphisms of a cytokine gene are associated with recurrent pregnancy loss. We propose that polymorphisms of IL1B may influence Th1-type immunity to trophoblast-specific proteins. Because IL-1a and IL-1b are important co-stimulators of Th2 cell proliferation²⁰⁻²² through an IL-4-independent pathway 22, they may be involved in the regulation of Th1 and Th2-type cytokine production. In this regard, production of IL-1 in the decidua may favor Th2 cell generation. Defective production of Th2 cytokines and leukemia inhibitory factor by decidual T cells in women with RPL has been reported⁷. In corroboration with this, a decrease in the endometrial IL-1b mRNA levels during the implantation window and early pregnancy was also observed in women with RPL²⁹. Thus, the association between IL1B-511C and RPL suggests to us that low IL-1b production at the maternal-fetal interface may reduce Th2-type cytokine production, and thus favor a biased Th1-type immune response to the developing conceptus, which may contribute to early pregnancy loss. A decreased IL-1b production might also affect trophoblast growth and invasion in early pregnancy.

Other genetic and environmental factors that influence Th1 cellular immunity may be involved in the mechanisms described above. This possibility is supported by a recent observation made in our laboratory that the mRNA levels of IL-12, an important cytokine for Th1-type immunity were significantly increased, and the mRNA levels of the Th3-type cytokine TGF-b were decreased in the decidua of women with RPL. These alterations, together with low IL-1 production, may contribute to an environment that does not favor conceptus development.

The precise mechanism(s) whereby polymorphism of the IL1B gene regulates Th1 and Th2-type cytokine production at the maternal-fetal interface remain to be defined. Our preliminary results indicate that trophoblast extracts do not induce IL-1b production in vitro by PBMC in women with RPL or in fertile controls, suggesting that the immunoregulatory effects of IL-1b on trophoblast-induced cytokine production might occur in the decidua. Further studies are needed to determine the relationship between IL1B polymorphism and production of IL-1b ,and Th1/Th2-type cytokines in the decidua of women with and without unexplained RPL. Our data suggest that genetic factor(s) are involved in the regulation of Th1 and Th2-type cytokine response in reproduction and that variants IL1B-511C and IL1B-31T confers susceptibility to RPL potentially mediated by Th1-type immunity to trophoblast.

### Example 1 Methods

Subjects. Study subjects were recruited from the Recurrent Pregnancy Loss Clinic within the Center for Reproductive Medicine at Brigham and Women's Hospital. Forty-eight women with a history of three or more first trimester spontaneous abortions of unexplained etiology who had evidence of Thl-type immunity to trophoblast as determined by PBMC IFN-g production⁴ and 46 RPL patients without evidence of Th1-type immunity to trophoblast were initially studied. Additional 42 women with unexplained RPL who had evidence of Th1-type immunity to trophoblast were subsequently recruited for verification. Additional 14 women with unexplained RPL were recruited to determine whether our observations were trophoblast-specific. All study subjects had a diagnosis of first trimester RPL of unknown etiology following clinical evaluation and laboratory tests indicating normal parental karyotypes, uterine structure and luteal phase. In addition, all study subjects were negative for anti-phospholipids antibodies and for cervical cultures. There was also no history of autoimmunity or atopy and none had a recent infections or illness. Fertile controls consist of 61 Caucasian women who had at least two prior successful pregnancies and no history of pregnancy loss.

Cytokine release assay. PBMCs were isolated from peripheral blood by Ficoll gradient centrifugation. Release of IFN-g by PBMCs in response to cell-free protein extracts derived from a trophoblast lineage cell line Jeg-3 and from mixed PBMCs of five unrelated individuals was determined as described previously⁴. IFN-g concentration was determined by ELISA (Endogen, Cambridge, Mass.). Women were classified as Th1 positive if their IFN-g response was at least 50 pg/ml and two-fold over background (unstimulated) levels. To test IFN-g production in response to allogenic cells and PHA mitogen, PBMCs were cocultured with g-irradiated (5000 rad) pooled PBMCs or with PHA for 5 days and the supernatants were tested for the concentration of IFN-g.

DNA isolation and analysis for polymorphisms in first intron of the IFNG gene, in the TNFA promoter region and in the IL1B and IL1RN genes. DNA was isolated using the QIAamp DNA minikit (QIAGEN Inc., Valencia, Calif.) following the manufacturer's instructions. CA-repeat polymorphism in the first intron of IFNG was amplified by PCR using primers 5′TCACAATTGATTTTATTCTTAC 3′ (SEQ ID NO: 2) and 5′ TGCCTTCCTGTAGGGTATT 3′ (SEQ ID NO: 3). PCR products were size-fractionated on 6% polyacrylamide sequencing gel and then transferred onto nylon membrane (Amersham Life Science, UK). Each allele was detected by hybridization with the upstream primer as probe labeled using the ECL labeling and detection system (Amersham Life Science, UK). TNFA promoter polymorphism at positions 238 (A/G), −376 (A/G), −862 (A/C) and 856 (T/C) were analyzed by the sequence specific oligonucleotide probe (SSOP) method. The SSOPs were as follows: −238G, 5′ CGGAATCGGAGCAGGGAG 3′ (SEQ ID NO: 4); −238A, 5′ CGGAATCAGAGCAGGGAG 3′ (SEQ ID NO: 5); −376A, 5′CTGTCTGGAAATTAGAAGGA 3′ (SEQ ID NO: 6); 376G, 5′ CTGTCTGGAAGTTAGAAGGA 3′ (SEQ ID NO: 7); −856C, 5′CTTAACGAAGACAGGGCC 3′(SEQ ID NO: 8); −856T, 5′ TTAATGAAGACAGGGCCA 3′(SEQ ID NO: 9); −862C, 5′ ATGGGGACCCCCCCTTAA 3′ (SEQ ID NO: 10); 862A, 5′ATGGGGACCCCCACTTAA 3′ (SEQ ID NO: 11). Oligonucleotide probes were 5′ end-labeled in the presence of [g-32P]ATP and T4 polynucleotide kinase (New England Biolabs, Mass.) according to the manufacturer's instructions. The TNFA promoter region (−1107 to 66) was amplified with primers 5′GCTTGTGTGTGTGTGTCTGG 3′ (SEQ ID NO: 12) and 5′GGACACACAAGCATCAAGG 3′ (SEQ ID NO: 13) using a Taq PCR Core kit (QIAGEN) with Q solution according to the manufacturer's instructions. The amplification profile was as follows: denaturing at 94° C. for 3 min followed by 35 cycles of denaturing at 94° C. for 1 min, annealing at 60° C. for 1 min and extension at 72° C. for 1 min. Two ml of PCR products was subjected to dot blotting, hybridization with 32P-SSOPs and high stringency washing following the manufacturer's instructions (LifeCodes Corp., Stamford, Conn.) with limited modifications. Blots were washed at 59° C. for 30 min. Polymorphisms at positions 511, −31 and +3953 of IL1B and at 308 of the TNFA promoter were analyzed using PCR-restriction fragment length polymorphism (RFLP) method as described before ^{(13,25,26,30,31)} with modifications. The primers used for amplifications were as follows: 5′ TGGCATTGATCTGGTTCATC 3′ (SEQ ID NO: 14) and 5′ GTTTAGGAATCTTCCCACTT 3′ (SEQ ID NO: 15) for IL1B-511, 5′ TCATAGTTTGCTACTCCTTGC 3′ (SEQ ID NO: 16) and 5′ CAAAAAGCTGAGAGAGGAGGC 3′ (SEQ ID NO: 17) for IL1B-31, 5′ ACCCCACTCCCAGCTTCATCC 3′ (SEQ ID NO: 18) and 5′ CTTGTTGCTCCATATCCTGTCC 3′ (SEQ ID NO: 19) for IL1B+3953, and 5′ AGGCAATAGGTTTTGAGGGCCAT 3′ (SEQ ID NO: 20) and 5′TCCTCCCTGCTCCGATTCCG 3′ (SEQ ID NO: 21) for TNFA-308. Amplification was performed in the presence of TaqGold polymerase (Perkin-Elmer, Norwalk, Conn.). The amplification profile was as follows: denaturing of DNA and activating enzyme at 95° C. for 10 min followed by 35 cycles of denaturing at 95° C. for 45 sec, annealing at 57° C. for 50 sec, and extension at 72° C. for 45 sec. PCR products for the IL1B and TNFA promoter regions were digested with restriction enzymes and analyzed by gel electrophoresis as previously described (13,25,26,30,31). Tandem repeat polymorphism in the second intron of IL1 RN was analyzed as described elsewhere (14).

Statistical analysis. Data were statistically analyzed by Fisher's Exact test (two tailed) with the aid of INSTAT software (GraphPad, San Diego, Calif.) or by Kruskal-Wallis one- way ANOVA and the Mann-Whitney rank sum test. Levels of significance were reported as P values (P<0.05 considered statistically significant).

### Example 1 References

1. Regan, L., B M J. 302, 543-544 (1991).

2. Hill, J. A. in Maternal-Fetal Medicine. 4th Edition. (eds. Creasy, R & Resnik, R) 423-443 (WB Saunders Co, 1998).

3. Wegmann, T. G., et al., Immunol. Today 14, 353-356 (1993).

4. Hill, J. A., et al., JAMA 273, 1933-1936 (1995).

5. Raghupathy, R., et al., Cell. Immunol. 196,122-130 (1999).

6. Makhseed, M., et al., Am J Reprod Immunol, 42, 273-281 (1999)

7. Piccinni, M-P. et al., Nature Med. 4, 1020-1024 (1998).

8. Hill, J. A., et al., Am. J. Obstet. Gynecol. 166, 1044-1052 (1992).

9. Hill, J. A. in Cellular and Molecular Biology of the Maternal-Fetal Relationship. (eds. Chaouat, G & Mowbray, J. F) 269-275 (INSERM/John Libbey Eurotext, 1991).

10. Yui, J., et al., Placenta 15, 819-827 (1994).

11. Raghupathy, R., Immunol. Today 18, 478-482 (1997).

12. Awad, M, et al., Hum Immunol. 60, 343-346 (1999).

13. McGuire, W, et al., Nature 371, 508-510 (1994).

14. Nemetz, A. et al., Immunogenetics 49, 527-531 (1999).

15. Wilkinson, R. J., et al., J. Exp. Med. 189, 1863-1873 (1999).

16. Yamada, H., et al., Am. J. Obstet. Gynecol. 170,1339-1344 (1994).

17. Robertson, S. A, et al., Crit. Rev. Immunol. 14, 239-292 (1994).

18. Hunter, C. A, et al., J. Immunol. 155, 4347-4354 (1995)

19. Tominaga, K., et al., Int Immunol 12, 151-160 (2000).

20. Lichtman, A. H, et al., Proc. Natl. Acad. Sci. U.S.A. 85, 9699-9703 (1988).

21. Manetti, R. et al., Res. Immunol. 145, 93-100 (1994).

22. Huber, M. et al., Int. Immunol. 8, 1257-1263 (1996).

23. Simon, C., et al., Fertil Steril 70, 896-906 (1998).

24. Librach, C. L, et al., J. Biol. Chem. 269,493-500 (1994).

25. di Giovine, F.S., et al., Hum. Mol. Genet. 1, 450 (1993).

26. Pociot, F., et al., Eur. J. Clin. Invest. 22, 396-402 (1992).

27. Santtila, S., et al., Scand. J. Immunol. 47, 195-198 (1998).

28. El-Omar, E. M., et al., Nature 404, 398-402 (2000).

29. Von Wolff, M., et al., Mol. Hum. Reprod. 6, 627-634 (2000).

30. Wilson, A. G., et al., Hum. Mol. Genet. 1, 353 (1992).

31. Guasch, J. F., et al., Cytokine 8, 598-602 (1996).

TABLE 1
EXAMPLE 1 Allelic frequencies of IFNG gene and TNFA promoter
region polymorphisms in women with RPL and fertile controls.

| | Fertile Control | RPL Th1 (−) | RPL Th1 (+) | |
|---|---|---|---|---|
| | (n* = 38) | (n = 66) | (n = 50) | |
| | No.* (%) | No. (%) | No. (%) | |
| IFNG alleles | | | | |
| A4 | 1 (2.6) | 0 (0.0) | 1 (2.1) | |
| A5 | 3 (8.0) | 2 (3.3) | 3 (6.3) | |
| A6 | 20 (52.6) | 40 (60.0) | 26 (52.1) | |
| A7 | 14 (36.8) | 24 (36.7) | 20 (39.5) | |
| | | | RPL Th1 (+) | RPL Th1 (+) |
| | Fertile Control | RPL Th1 (−) | Group 1 | Group 2 |
| | (n = 80) | (n = 92) | (n = 96) | (n = 84) |
| | No. (%) | No. (%) | No. (%) | No. (%) |
| TNFA | | | | |
| promoter | | | | |
| variants | | | | |
| −308A | 12 (15.0) | 17 (18.4) | 15 (15.6) | — # |
| G | 68 (85.0) | 75 (81.6) | 81 (84.4) | — |
| −376A | 0 (0.0) | 0 (0.0) | 0 (0.0) | 0 (0.0) |
| G | 80 (100.0) | 92 (100.0) | 96 (100.0) | 84 (100.0) |
| −238A | 3 (3.7) | 4 (4.3) | 5 (5.2) | 6 (7.1) |
| G | 77 (96.3) | 88 (95.7) | 91 (94.8) | 78 (92.9) |
| −856T | 9 (11.5) | 7 (7.6) | 9 (9.4) | 6 (7.1) |
| C | 71 (88.5) | 85 (92.4) | 87 (90.6) | 78 (92.9) |
| −862A | 8 (10.0) | 9 (9.8) | 14 (14.5) | 10 (11.9) |
| C | 72 (90.0) | 83 (90.2) | 82 (85.5) | 74 (88.1) |

*number of alleles.
# not typed.

TABLE 2
EXAMPLE 1 Allelic distributions of IL1B and ILRN gene
polymorphisms in women with RPL and fertile controls.

| | Fertile | RPL | RPL Th1 (+) | RPL Th1 (+) |
|---|---|---|---|---|
| | Control | Th1 (−) | Group 1 | Group 2 |
| | (n# = 122) | (n = 92) | (n = 96) | (n = 84) |
| Alleles | No.# (%) | No. (%) | No. (%) | No. (%) |
| IL1B − 511 | | | | |
| C | 63 (51.6) | 56 (60.8) | 73 (76.0) 1,2 | 57 (67.8) 3 |
| T | 59 (48.4) | 36 (39.2) | 23 (24.0) 1,2 | 27 (32.2) 3 |
| IL1B + 3953 | | | | |
| C | 92 (75.4) | 72 (78.3) | 66 (68.8) | 58 (69.1) |
| T | 30 (24.6) | 20 (21.7) | 30 (31.2) | 26 (30.9) |
| IL1RN | | | | |
| *1 | 79 (64.7) | 61 (66.3) | 64 (68.1) | 56 (70.0) |
| *2 | 40 (32.8) | 27 (29.3) | 28 (29.8) | 21 (26.3) |
| *3 | 3 (2.5) | 2 (2.2) | 0 (0.0) | 2 (2.5) |
| *4 | 0 (0.0) | 2 (2.2) | 2 (2.1) | 1 (1.2) |

# number of alleles.
1 P = 0.0002, compared with fertile controls.
2 P = 0.028, compared with RPL Th1 (−) group.
3 P = 0.022, compared with fertile controls.

### Example 2 Description

### Genetic Interaction Between CD46 and IL1B alleles Influences T Helper 1-type Immunity to Trophoblast and Recurrent Pregnancy Loss

T helper (Th)1 -type immunity to trophoblast is associated with recurrent pregnancy loss (RPL). This response is determined by measuring interferon-gamma (IFN-γ) produced by peripheral blood mononuclear cells exposed to extracts of trophoblast. CD46 and interleukin(IL)-1 β are involved in the regulation of Th1/Th2 immunity and their genes are polymorphic. We detected a significantly higher frequency of homozygotes for two alleles of these genes (allele 2 at the loci of HindIII site in intron 1 of the CD46 gene and IL1B-511C, a variant of the IL1B promoter region) in women with RPL and Th1 immunity to trophoblast (RPL-Th1). The association between these two genotypes and RPL-Th1 was more significant when both homozygotes were present, suggesting a genetic “interaction” that predisposes women to Th1 immunity to trophoblast and RPL. We propose that determination of both homozygotes is useful for predicting Th1 immunity to trophoblast in women with RPL.

Recurrent pregnancy loss (RPL) is an important women's health problem affecting approximately 1 in 300 pregnancies. Its etiology is enigmatic in about 50% of cases. T helper (Th) 1-type immunity may contribute to reproductive failure since Th1 -type cytokines such as interferon-gamma (IFN-γ) and tumor necrosis factor-alpha (TNF-α) may disrupt a number of normal reproductive processes.^{1,2} T helper (Th) 1 -type immunity to trophoblast antigens and defective Th2 -type cytokine production by decidual T cells have been associated with RPL.^{3,4} Human CD46, a membrane cofactor protein of the complement system, and IL-1 have been demonstrated to play a role in regulation of Th1/Th2 immunity.^{5,6} The genes for CD46 and IL-1β are polymorphic. There is a biallelic polymorphism at restriction enzyme HindIII site in the first intron of the CD46 gene and at position −511 of the IL1B promoter region (IL1B-511).^{7,8} We investigated the association of polymorphism of the CD46 gene and IL1B promoter region with RPL and Th1 immunity to trophoblast, and explored the potential genetic interaction of the two genes in RPL.

We performed a case-controlled study in 131 Caucasian women previously evaluated in the Recurrent Pregnancy Loss Clinic within the Center for Reproductive Medicine at Brigham and Women's Hospital. They had a history of at least three or more first trimester spontaneous abortions without defined etiology following a thorough clinical evaluation and laboratory testing, including normal parental chromosomes, a normal uterine structural study and endometrial biopsy, negative cervical cultures (Chlamydia, Mycopalsma, Ureaplasma, and Group B Streptococcus), and negative anti-phospholipid antibodies. None of these women had a history of recent infection or autoimmunity. These women had been previously tested for Th1 immunity to trophoblast as described previously 3 by assaying IFN-γ production by peripheral blood mononuclear cells (PBMCs) stimulated with a protein extract (30 μg/ml) derived from the trophoblast cell line Jeg-3. Previous studies demonstrated that IFN-γ production in this in vitro assay was associated with RPL, since fertile women seldom had a positive IFN-γ response to trophoblast (less than 3%).³ Women whose PBMCs produced at least 50 pg/ml of IFN-γ and two fold over background (not stimulated) levels were designated as the RPL Th1(+) subgroup (mean IFN-γ level 200.1±27 pg/ml). Women in the RPL Th1 (−) subgroup did not produce IFN-γ over background levels. The Th1(+) and Th1 (−) subgroups were comparable in age (range: 24-42 years) and number of losses [mean 3.873, range 3-11 for Th1(+) groups and mean 3.95, range 3-7 for Th1(−) groups]. Seventy two Caucasian women who had a history of at least two prior successful pregnancies with no history of pregnancy loss served as fertile controls. Polymorphism of CD46 and IL1B genes was typed using polymerase chain reaction-restriction fragment length polymorphism method.^{7,8} Data were statistically analyzed by Fisher's Exact test (two tailed). Levels of significance were reported as Pc derived from P value corrected by multiple comparisons to overcome the potential statistical type I error. Alleles at HindIII site in intron 1 of the CD46 gene and of IL1B-511 in our fertile women group were in Hardy-Weinberg equilibrium.

The allele frequency of IL1B-511C was significantly increased (Pc<0.0008) in the Th1(+) RPL group when compared with fertile controls (Table 1). In addition, the frequency of allele 2 at the loci of HindIII site in intron 1 of the CD46 gene (CD46 allele 2) was also increased in the Th1(+) RPL group, although the difference did not reach statistical significance (Pc=0.07, Table 1). Moreover, there was also a higher frequency of homozygotes for these two alleles in the Th1(+) RPL group. However, the frequency of these genotypes was not significantly different between Th1(−) RPL group and fertile controls. These results suggest an association between RPL with Th1 immunity to trophoblast and homozygosity for CD46 allele 2 and/or IL1B-511C.

We further determined whether homozygotes for IL1B-511C and CD46 allele 2 had a combination effect on the risk for RPL associated with Th1 immunity to trophoblast using a method based on two-by-two tests of various genotype comparisons as previously described by Svejgaard A and Ryder LP.⁹ We assumed that homozygosity for IL1B-511C (genotype A) and CD46 allele 2 (genotype B) were risk factors for RPL associated with Th1 immunity to trophoblast. Although these genotypes alone were found more frequently in the Th1 (+) RPL group than in fertile controls [odds ratios (OR) of 3.4 and 4.4, respectively], women with both genotypes had a higher risk for RPL associated with Th1 immunity to trophoblast as compared to absence of these two genotypes (OR=11.6; Pc<0.0016; Table 2b), indicating a combination effect of these two genotypes. This effect appeared specific, since it did not occur in the Th1(−) RPL group in comparison with fertile controls. Further analysis with various comparisons between Th1 (+) RPL and Th1 (−) RPL groups suggested that genotype A and B alone were not as significant indicators of Th1 immunity to trophoblast following correction of the P values for multiple comparisons; However, the risk for Th1 immunity to trophoblast was increased in the presence of both genotypes, and the increase was statistically significant when compared to the absence of these genotypes (OR=9.45; Pc=0.029; Table 2b). Moreover, out of 18 women with a history of RPL who were homozygous for both IL1B-511C and CD46 allele 2, 16 were Th1 (+) but only 2 were Th1(−) (88.9% vs. 11.1%; P <0.0001; OR=64; 95%CI: 8.001-511.96). Thus, combination of the two homozygotes had the strongest association with Th1 immunity to trophoblast in women with RPL. These results suggested an interaction between CD46 and IL1B alleles in induction of Th1 immunity to trophoblast.

The genetic interaction of two genes on different chromosomes represents a new polygenic model for a genetic influence on regulation of Th1/Th2 immune responsiveness in reproduction. Only homozygotes for the two predisposing alleles conferred the susceptibility to RPL, suggesting a low functional phenotype of these alleles. Functionally, IL-1 may affect Th2 cell generation,⁶ while CD46 may inhibit IL-12 production in a Th1 response. In this regard, polymorphism of CD46 and IL1B genes may synergize to produce susceptibility to RPL associated with Th1 immunity to trophoblast. The combination of homozygotes for CD46 allele 2 and IL1B-51 C appears valuable for predicting Th1 immunity to trophoblast in women with RPL and may ultimately be usefull for the clinical management of this disorder.

### Example 2 References

1. Hill J A, et al., Am. J Obstet. Gynecol. 1992; 166: 1044-52.

2. Yui J, et al., Placenta 1994; 15:819-27.

3. Hill J A, et al., JAMA 1995; 273: 1933-36.

4. Piccinni M-P, et al., Nature Med 1998; 4:1020-24.

5. Karp C L, et al., Science 1996; 273:228-31.

6. Manetti R., et al., Res Immunol. 1994; 145: 93-100.

7. Bora N S, et al., J Immunol. 1991; 146:2821-25.

8. di Giovine F S, et al., Hum. Mol. Genet. 1993; 1:450.

9. Svejgard A, and Ryder L P. Tissue Antigens. 1994; 43:18-27.

TABLE 1
Example 2 Polymorphism at the HindIII site in intron 1 of
the CD46 gene and variants at position −511 of the IL1B
promoter region in women with a history of RPL and fertile controls.

| | Th1 (+) RPL | | Th1 (−) RPL | | | |
|---|---|---|---|---|---|---|
| Fertile | (n^{a} = 72) | (n = 71) | | | (n = 60) | |
| Control | No. (%) | No. (%) | P^{b} | Pc^{c} | No. (%) | P^{b} |
| Allele | | | | | | |
| frequency | | | | | | |
| CD46 | 92 (63.9) | 68 (47.9) | | | 69 (57.5) | |
| allele 1 | | | | | | |
| allele 2 | 52 (36.1) | 74 (52.1) | 0.0087 | 0.07 | 51 (42.5) | NS^{d} |
| IL1B-511 | 74 (51.4) | 109 (76.8) | <0.0001 | <0.0008 | 70 (58.3) | |
| C | | | | | | |
| T | 70 (48.6) | 33 (23.2) | | | 50 (41.7) | |
| Genotype | | | | | | |
| CD46 | 27 (37.5) | 20 (28.2) | NS | | 22 (36.7) | NS |
| allele 1,1 | | | | | | |
| allele 2,2 | 7 (9.7) | 23 (32.4) | 0.001 | 0.012 | 13 (21.6) | NS |
| allele 1,2 | 38 (52.7) | 28 (39.4) | NS | | 25 (41.7) | NS |
| IL1B-511 | 21 (29.2) | 42 (59.2) | 0.0004 | 0.0048 | 23 (38.3) | NS |
| CC | | | | | | |
| TT | 19 (26.4) | 4 (5.6) | 0.0011 | 0.013 | 13 (21.7) | NS |
| CT | 32 (44.4) | 25 (35.2) | NS | | 24 (40.0) | NS |

^{a}Case number.
^{b}P value for comparison with fertile controls.
^{c}P value corrected by the number of comparisons (P × 8 for allele frequency and P × 12 for genotype).
^{d}P > 0.05 (not statistical significant).

TABLE 2a
Example 2, Genotypes of IL1B-511 and CD46
in women with a history of RPL

| | CD46 2, 2 | | Number (percent) | |
|---|---|---|---|---|
| ILB-511CC | (genotype | Fertile | Th1 (+) | Th1 (−) |
| (genotype A) | B) | (n = 72) | RPL (n = 71) | RPL (n = 60) |
| + | + | 3 (4.2) | 16 (22.5) | 2 (3.3) |
| + | − | 17 (23.6) | 26 (36.6) | 21 (35) |
| − | + | 4 (5.6) | 7 (9.9) | 11 (18.3) |
| − | − | 48 (66.7) | 22 (31) | 26 (43.3) |

Example 2, Table 2b. Two-by-two comparisons.

| Comparison | a | b | c | d | OR | P | Pc (P × 16) |
|---|---|---|---|---|---|---|---|
| Th1(+) RPL vs. Fertile: | | | | | | | |
| A vs. non-A | 42 | 29 | 22 | 52 | 3.4 | 0.0004 | 0.0064 |
| B vs. non-B | 23 | 48 | 7 | 65 | 4.4 | 0.001 | 0.016 |
| AB AB | | | | | | | |
| ++ vs. −+ | 16 | 7 | 3 | 4 | 3.1 | 0.370 | |
| +− vs. −− | 26 | 22 | 17 | 48 | 3.3 | 0.0033 | 0.053 |
| ++ vs. +− | 16 | 26 | 3 | 17 | 3.5 | 0.082 | |
| −+ vs. −− | 7 | 22 | 4 | 48 | 3.8 | 0.048 | 0.768 |
| ++ vs. −− | 16 | 22 | 3 | 48 | 11.6 | <0.0001 | <0.0016 |
| +− vs. −+ | 26 | 7 | 17 | 4 | 0.9 | >0.9999 | |
| Th1(−)RPL vs. Fertile: | | | | | | | |
| A vs. non-A | 23 | 37 | 3 | 4 | 1.5 | 0.450 | |
| B vs. non-B | 13 | 47 | 7 | 65 | 2.6 | 0.240 | |
| AB AB | | | | | | | |
| ++ vs. −+ | 2 | 11 | 3 | 4 | 0.2 | 0.289 | |
| +− vs. −− | 21 | 26 | 17 | 48 | 2.3 | 0.046 | 0.730 |
| ++ vs. +− | 2 | 21 | 3 | 17 | 0.5 | 0.650 | |
| −+ vs. −− | 11 | 26 | 4 | 48 | 5.07 | 0.0091 | 0.146 |
| ++ vs. −− | 2 | 26 | 3 | 48 | 1.2 | >0.9999 | |
| +− vs. −+ | 21 | 11 | 17 | 4 | 0.5 | 0.351 | |
| Th1(+) RPL vs. | | | | | | | |
| Th1(−)RPL: | | | | | | | |
| A vs. non-A | 42 | 29 | 23 | 37 | 2.3 | 0.023 | 0.368 |
| B vs. non-B | 23 | 48 | 13 | 47 | 1.7 | 0.240 | |
| AB AB | | | | | | | |
| ++ vs. −+ | 16 | 7 | 2 | 11 | 12.57 | 0.0045 | 0.072 |
| +− vs. −− | 26 | 22 | 21 | 26 | 1.5 | 0.414 | |
| ++ vs. +− | 16 | 26 | 2 | 21 | 6.5 | 0.018 | 0.288 |
| −+ vs. −− | 7 | 22 | 11 | 26 | 0.8 | 0.781 | |
| ++ vs. −− | 16 | 22 | 2 | 26 | 9.45 | 0.0018 | 0.029 |
| +− vs. −+ | 26 | 7 | 21 | 11 | 1.9 | 0.277 | |

### Example 3 Description

### T Helper 1-type Immunity to Trophoblast Antigens in Women with a History of Recurrent Pregnancy Losses is Associated with Polymorphism of the IL1B Promoter Region

Recurrent pregnancy loss (RPL) is a common disorder during early gestation. Recent evidence suggests that T helper 1 (Th1)-type immunity is associated with unsuccessful pregnancy especially in women with RPL of otherwise unknown etiology, while Th2-type immunity is associated with pregnant success. Interleukin (IL)-1 may influence Th1/Th2 immune responsiveness and has been implicated in the establishment of successful pregnancy. In the present study, we investigated polymorphism of the IL-1 p gene (IL1B) in women with a history of RPL. A significant increase in the frequency of IL1B promoter region variants IL1B-511C and IL1B-31T was found in women with a history of RPL. The increase of the frequency of these two variants and their homozygotes was found only in the cases having evidence of Th1 immunity to trophoblast as determined by IFN-γ production of peripheral blood mononuclear cells (PBMCs) stimulated with a trophoblast cell-line extract. Significantly higher IFN-γ production by PBMCs in response to trophoblast correlated with variant IL1B-511C and its homozygocity in women with RPL. These results suggest that variants -511C and -31T in the IL1B promoter region may confer risk for RPL associated with Th1 immunity to trophoblast antigens.

Recurrent pregnancy loss (RPL), defined as three or more first trimester spontaneous abortions, is an important women's health problem. In approximately 50% of cases the etiology remains unknown.^{1,2} Recent studies in rodents provided evidence indicating that predominant T helper 2 (Th2)-type cytokine production at the fetal-maternal interface is associated with successful gestation; while Th1-type immunity is incompatible with pregnancy success. ^{3,4} Studies in women have suggested that Th1-type immunity to trophoblast antigens^{5,6} and defective Th2 cytokine production by decidual T cells were associated with RPL.⁷ Th1-type immunity may contribute to reproductive failure, since Th1 cytokines such as interferon-gamma (IFN-γ) and tumor necrosis factor-alpha (TNF-α) have been demonstrated to disrupt a number of reproductive process as proved in vitro.^{8,9} The potential molecular and genetic mechanism(s) underlying these phenomena remains unknown. A growing body of evidence has shown that polymorphism of cytokine genes influences cytokine production and may be associated with susceptibility to certain infectious, inflammatory and autoimmune diseases.¹⁰⁻¹⁴ However, our preliminary studies have not revealed an association between RPL and polymorphism of the genes for Th1 cytokines (IFN-γ and TNF-α).

Cytokines in the IL-1 system [IL-1α, IL-1β and IL-1 receptor antagonist (IL-1Ra)] are produced at the maternal-fetal interface during early pregnancy.¹⁵ The IL1 system may be involved in the regulation of Th1/Th2 cytokine production, since IL-1 can function as a co-stimulator for Th2 cell generation in both rodents and human¹⁶⁻¹⁹ and may influence IFN-γ production mediated by natural killer (NK) and T cells.^{20 21} Moreover, IL-1 has been implicated in implantation,²² trophoblast growth and invasion.²³ It was reported that mRNA for IL-1β in the endometrium was decreased in women with a history of recurrent pregnancy loss.²⁴ The genes of the IL-1 system are polymorphic. Biallelic base substitution (C to T) polymorphism has been described at positions −511, −31 and +3953 bases away from the transcriptional start site of the IL-1β gene (IL1B).^{14,25,26} There is also a tandem repeat polymorphism in the IL-1Ra gene (IL1RN).¹⁰ Recent studies have shown that polymorphism of these genes influences cytokine production and susceptibility to certain infectious, inflammatory and malignant diseases.¹²⁻¹⁴ A recent study reported an association between IL1RN polymorphism and recurrent pregnancy loss.²⁷ In the present study, we investigated polymorphisms of the IL1B gene and Th1 cytokine production in women with RPL.

We performed a retrospective case-controlled study in Caucasian women recruited at Brigham and Women's Hospital between November 1995 and July 1996 who attended the Recurrent Pregnancy Loss Clinic within the Center for Reproductive Medicine.

All these women had a history of three or more first trimester spontaneous abortions (many up to nine times) and had been diagnosed as having RPL of unexplained etiology following a thorough clinical evaluation and laboratory testing involving negative cervical cultures for organisms associated with pregnancy loss (Chlamydia, Mycopalsma, Ureaplasma, and Group B Streptococcus), negative anti-phospholipids antibodies, normal parental karyotypes, normal hormone testing and a normal intrauterine cavity. In addition, none of these women had a history of a recent infection or had a history of autoimmunity. Additional healthy Caucasian women who had a history of at least two successful pregnancies with no pregnancy loss served as fertile controls. Alleles at the loci of IL1B in our fertile women group were in Hardy-Weinberg equilibrium. To address a possible statistical type I error for our first database, another separate group of Caucasian women with a history of RPL (recruited from our Clinic between August 1996 and March 1997) and fertile Caucasian women was subsequently included in a second database. These women met the criteria described above. All of the women with a history of RPL in our two databases had been routinely tested for Th1 immunity to trophoblast as described previously⁵ by assaying IFN-γ produced by peripheral blood mononuclear cells (PBMCs) stimulated with a protein extract derived from the trophoblast lineage cell line Jeg-3 (Ref 28). This cell line is endocrinologically and antigenically similar to normal invasive trophoblasts.^{29,30} Peripheral blood samples were collected before any medications were given while not pregnant and at least two normal menstrual cycles had occurred since the last spontaneous abortion. Using this assay we have previously demonstrated higher production of the Th1 type cytokine IFN-γ and lower production of the Th2 cytokine IL-10 by PBMCs in a subgroup of women with unexplained RPL.^{5,6} Less than 3% of fertile women had a positive IFN-γ response in this assay system. Based on PBMC IFN-γ production to trophoblast antigen challenge, we divided these women with RPL into Th1(+) and Th1 (−) subgroups which were similar in age (range: 24-42 years) and number of losses [mean 3.873, range 3-11 for Th1(+) groups and mean 3.95, range 3-7 for Th1(−) groups]. Women whose PBMCs produced at least 50 pg/ml of IFN-γ and two-fold over background (not stimulated) levels following exposure to trophoblast extracts in vitro were designated as the RPL Th1 (+) subgroup. The mean IFN-γ level of the RPL Th1 (+) group was 215.49±34 pg/ml in the first database series and 165.4±35.4 pg/ml in the second database series. Women in the RPL Th1 (−) subgroup did not produce IFN-γ over background levels.

Our initial experiments using restriction fragment length polymorphism (RFLP) analysis for IL1B polymorphism revealed that the frequency of the variant IL1B-511C of the IL1B promoter region was significantly higher in women with a history of RPL, compared with fertile controls in the first database series (66.9% vs. 50%, respectively; P=0.045; Table 1). We reconfirmed this difference between another RPL group and fertile controls in separate experiments (69.4% vs. 52.3%, respectively; P=0.011; Table 1). The frequency of the variant IL1B-31T was also increased in the RPL groups. There was no significant difference in the distributions of the alleles of IL1B at position +3953 and of IL1RN among our study groups. These data suggest that polymorphism of the IL1B promoter region may be associated with RPL.

We further determined whether the increased frequency of variants IL1B-511C and IL1B-31T in women with a history of RPL was associated with Th1 immunity to trophoblast. Analysis in the first database series revealed a significantly increased frequency of IL1B-511C in the RPL Th1(+) group, compared with fertile controls (72.9% vs. 50%, respectively; P=0.011; Table 2). Furthermore, a similar increase in the frequency of the IL1B-31T in the RPL Th1(+) group was also found (71.4% vs. 48.3%, respectively; P=0.029; Table 2). Thus, we hypothesized that IL1B polymorphism was associated with Th1 immunity to trophoblast. To test this hypothesis, we analyzed a second database series, and again found a significantly increased frequency of IL1B-511C (80.6% vs. 52.3%, respectively; P=0.0003) and IL1B-31T (79.2% vs 52.3%, respectively; P=0.0007) in the Th1(+) group, compared with fertile controls (Table 2). However, we observed no significant difference in the IL1B-511C and IL1B-31T allele frequency between the RPL Th1(−) group and fertile controls in both our database series.

The increased frequency of these variants in the RPL Th1(+) groups correlated with an increased frequency of their homozygosity. There was an increased frequency of homozygotes for IL1B-511C and IL1B-31T in Th1(+) RPL group in both database series, compared with fertile controls (Table 2). We also observed a decreased frequency of the homozygotes for variant IL1B-511T in the RPL Th1(+) groups (Table 2). No significant difference in the IL1B-511C and IL1B-31T allele frequency or their homozygotes was found between either of our RPL Th1(−) groups and fertile controls. These results suggest that homozygosity for IL1B-511C and IL1B-31T confers susceptibility to RPL associated with Th1-type immunity to trophoblast.

In our fertile controls and study groups, the alleles at IL1B-511 were in strong linkage disequilibrium with those at IL1B-31T, since more than 91% of cases being homozygous for IL1B-511C were also homozygous for IL1B-31T. The same was true for IL1B-511T and IL1B-31C homozygotes and for heterozygotes of these variants.

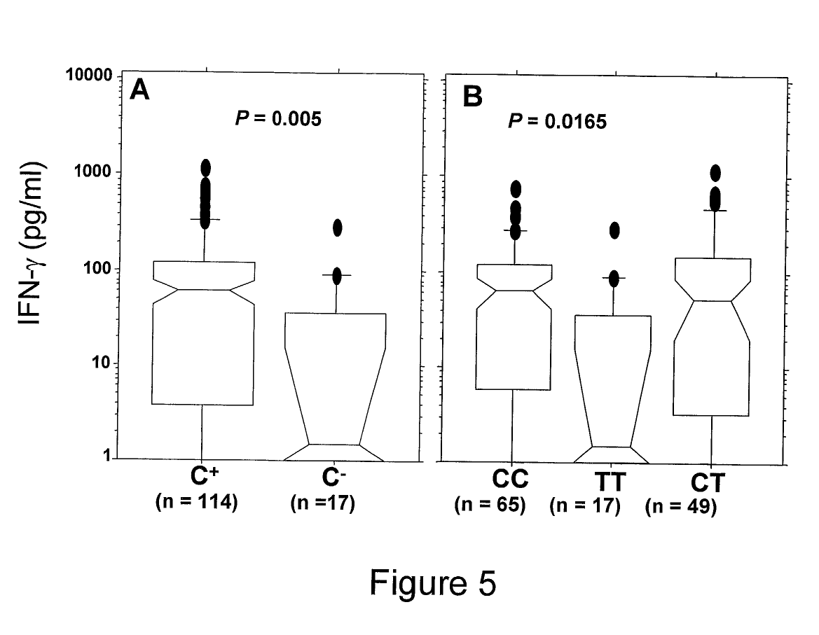

We next analyzed the relationship between variants at IL1B-511 and trophoblast extract-induced IFN-γ production by PBMCs in women with RPL. As shown in FIG. 5A, high production of IFN-γ occurred more frequently in IL1B-511C carriers (C+) than in IL1B-511C noncarriers (C⁻) who had RPL (median level 60.2 pg/ml vs. 1.5 pg/ml, respectively; P=0.005). The IL1B-511C/C genotype was associated with high IFN-γ production in response to trophoblast antigens, compared with the IL1B-511T/T genotype in women with RPL (median level 63 pg/ml vs. 1.5 pg/ml, respectively; P=0.0165; FIG. 1B). IFN-γ production was also increased in the individuals with the IL1B-511C/T genotype, compared to those with IL1-511T/T (median level 43.2 pg/ml vs. 1.5 pg/ml). These results are consistent with our hypothesis that polymorphisms of the IL1B promoter region influences Th1 immunity to trophoblast antigens in women with RPL.

To further evaluate the specificity of IFN-γ response to trophoblast extracts, we challenged PBMCs from a group of 20 women with a history of RPL with a protein extract from Jeg-3 cells, red blood cell membrane and allogenic PBMC extracts. We found that red blood cell membrane and allogenic PBMC extracts did not induce IFN-γ production in any of the cases, whereas 7 cases demonstrated positive IFNy production following exposure to extracts of trophoblast cell line Jeg-3. Furthermore, there was also no correlation between the levels of IFNγ production in trophoblast-stimulated cultures and these of PHA or mixed lfymphocytes-stimulated cultures. Thus, IFN-γ production in this assay appears to be a response to trophoblast antigens. We also compared PBMC IFN-γ production to Candida Albicans extracts (recall antigen) vs. trophoblast extract in a separate group of women with RPL (n=25). We found that Candida antigen extracts induced IFN-γ in all cases except one (median 473 pg/ml, range 0 to 1290, in 18 women who did not have Th1 immunity to trophoblast; median 384 pg/ml, range 197 to 1253, in 7 women who had Th1 immunity to trophoblast), suggesting that Th1 immunity to trophoblast does not correlate with that to Candida antigens. In addition, Th1 cytokine production by PBMCs, including in response to trophoblast extracts, was not influenced by the menstrual cycle 31, although the levels of IL-4 produced by PBMCs and IL-1 and IL-8 in serum may vary in the two phases of menstrual cycles.^{31,33} Therefore, Th1 immunity to trophoblast, which is associated with IL1B polymorphism in women with RPL, appears to be a specific memory immune response.

To our knowledge, this is the first study demonstrating that polymorphisms of a cytokine gene are associated with Th1 immunity to trophoblast in women with RPL. IL1B polymorphisms have been associated with certain infectious, inflammatory and autoimmune diseases. However, in this study there was no evidence of infections clinically or by history, cultures and endometrial histology. Similarly, inflammation was not found histologically in spontaneously aborted tissues from these women. Recent observations of decreased endometrial IL-1β mRNA levels in the menstrual cycle ²⁴ and during early pregnancy in women with RPL of unknown etiology also do not suggest the involvement of infection or a pro-inflammatory response.

It was reported that homozygotes for IL1B-511C had lower IL-1P production by monocytes stimulated with PMA or antigens, compared with IL1B-511T homozygotes.^{13,34} Although no direct information about IL1B-31T in IL-1β production currently exists, the association of homozygosity for IL1B-511C and IL1B-31T with RPL is consistent with the phenotype of low IL-1 production. These variants may theoretically favor reduced endometrial IL-1β mRNA levels which was observed in women with RPL.²⁴ Altered IL-1 production may affect the balance of Th1/Th2 cytokine responsiveness, involving either pregnancy success or failure by two potential mechanisms. First, since IL-1α and IL-1β are important co-stimulators for Th2 cell proliferation, ¹⁶⁻¹⁹ decidual IL-1 may influence Th2 cell generation. Conversely, low IL-1β production at the maternal-fetal interface may reduce Th2-type cytokine production, and thus, in concert with elevated decidual IL-12 levels, favor a Th 1-type response to the developing conceptus, which could contribute to early pregnancy failure. This potential mechanism is supported by recent observations in our laboratory of a correlation between reduction of IL-1β and elevated IFN-γ/IL-10 ratio at the mRNA level in the decidua from failed pregnancy associated with a chromosomally normal. Altered IL-1β production in the decidua may explain defective production of Th2 cytokines by decidual T cells recently reported in women with RPL.⁷ A second mechanism whereby altered IL-1β production may culminate in pregnancy loss could be by directly affecting trophoblast growth and invasion during early pregnancy. These mechanisms are distinct from those in other diseases associated with over-production and the pro-inflammatory effect of IL-1. ^{12,14}

Other gene(s) in linkage disequilibrium with IL1B-511C and IL1B-31T also play a role. (See Example 2). Our data suggest that genetic factors are involved in the regulation of Th1 and Th2-type cytokine responsiveness in reproduction, and that homozygosity for IL1B-511C and IL1B-31T confers susceptibility to RPL associated with Th1-type immunity to trophoblast. Further studies are needed to define the relationship between IL1B polymorphism, production of IL-1β, and Th1/Th2-type cytokines in the decidua of women with unexplained RPL.

Example 3, Table 1. Allele distributions of ILIB and ILRN
gene polymorphism in women with RPL and fertile controls.

| | First Database | | | Second Database | | |
|---|---|---|---|---|---|---|
| | Fertile | RPL | | Fertile | RPL | |
| | (n# = 60) | (n = 118) | | (n = 84) | (n = 144) | |
| Alleles | No.# (%) | No. (%) | P | No. (%) | No. (%) | P |
| IL1B-511 | | | | | | |
| C | 30 (50.0) | 79 (66.9) | 0.035 | 44 (52.3) | 100 (69.4) | 0.011 |
| T | 30 (50.0) | 39 (33.1) | | 40 (47.7) | 44 (30.6) | |
| IL1B-31 | | | | | | |
| C | 31 (51.7) | 43 (36.4) | | 40 (47.7) | 46 (31.9) | |
| T | 29 (48.3) | 75 (63.6) | 0.056 | 44 (52.3) | 98 (68.1) | 0.023 |
| IL1B + 3953 | | | | | | |
| C | 46 (76.5) | 85 (72.1) | 0.59 | 59 (70.2) | 106 (73.6) | 0.64 |
| T | 14 (23.5) | 33 (27.9) | | 25 (29.8) | 38 (26.4) | |
| IL1RN | | | | | | |
| *1 | 39 (65.0) | 88 (74.6) | 0.22 | 60 (71.4) | 99 (68.7) | 0.77 |
| *2 | 20 (33.3) | 26 (22.0) | 0.10 | 22 (26.2) | 40 (27.8) | 0.56 |
| *3 | 1 (3.3) | 2 (1.7) | 1.00 | 2 (2.4) | 2 (1.4) | 0.59 |
| *4 | 0 (0.0) | 2 (1.7) | 0.55 | 0 (0.0) | 3 (2.1) | 0.31 |
| DNA was isolated from PBMCs of study and control subjects using the QIAamp DNA minikit (QIAGEN Inc., Valencia, CA). Alleles at the loci of IL1B were typed using polymerase chain reaction (PCR)-RFLP methods as described elsewhere.^{25, 26, 35} Tandem | | | | | | |
| # repeat polymorphism in the second intron of IL1RN was analyzed by PCR-gel electrophoresis as described.¹³ # number of alleles. Data were statistically analyzed by Fisher's Exact test (two tailed) with the aid of INSTAT (GraphPad, San Diego, CA). | | | | | | |
| # Levels of significance were reported as P values (P < 0.05 considered statistically significant). | | | | | | |

Example 3, Table 2. Allele frequency and genotypes of variants in the IL1B
promoter region in women of Thl(+) RPL and Thl(−) RPL subgroups.

| | First Database | | | Second Database | | |
|---|---|---|---|---|---|---|
| | Fertile | Thl(+) RPL | Thl(−) RPL | Fertile | Thl(+) RPL | Thl(−) RPL |
| | No. (%) | No. (%) | No. (%) | No. (%) | No. (%) | No. (%) |
| Allele | (2n* = 60) | (2n = 70) | (2n = 48) | (2n = 84) | (2n = 72) | (2n = 72) |
| frequency | | | | | | |
| IL1B-511 | | | | | | |
| C | 30 (50.0) | 51 (72.9)^{a} | 28 (58.4) | 44 (52.3) | 58 (80.6)^{b} | 42 (58.3) |
| T | 30 (50.0) | 19 (27.1) | 20 (41.6) | 40 (47.7) | 14 (19.4) | 36 (41.7) |
| IL1B-31 | | | | | | |
| C | 31 (51.6) | 20 (28.6) | 23 (47.9) | 40 (47.7) | 15 (20.8) | 31 (43.1) |
| T | 29 (48.3) | 50 (71.4) | 25 (52.1) | 44 (52.3) | 57 (79.2) | 41 (56.9) |
| Genotypes | (n#= 30) | (n = 35) | (n = 24) | (n = 42) | (n = 36) | (n = 36) |
| IL1B-511 | | | | | | |
| CC | 8 (26.7) | 18 (51.4)^{c} | 10 (41.7) | 13 (30.9) | 24 (66.7)^{f} | 13 (36.2) |
| TT | 8 (26.7) | 2 (5.7)^{a} | 6 (25.6) | 11 (26.2) | 2 (5.6)^{h} | 7 (19.4) |
| CT | 14 (46.6) | 15 (42.9) | 8 (33.3) | 18 (42.9) | 10 (27.7) | 16 (44.4) |
| IL1B-31 | | | | | | |
| CC | 8 (26.7) | 2 (5.7)ⁱ | 7 (29.2) | 11 (26.2) | 3 (8.3)ⁱ | 7 (19.4) |
| TT | 7 (23.3) | 17 (48.6)^{k} | 8 (33.3) | 13 (30.9) | 24 (66.7)^{l} | 12 (33.3) |
| CT | 15 (50.0) | 16 (45.7) | 9 (37.5) | 18 (42.9) | 9 (25.0) | 17 (47.2) |

*Total allele number. # Total case number. P = 0.011, odds ratio (OR): 2.684; 95% confidence interval (CI): 1.293-5.574. ^{b}P = 0.0003, OR: 3.766, 95% IC: 1.826-7.769.
^{c}P = 0.029, OR: 2.339, 95% CI: 1.113-4.829. ^{d}P 0.0007, OR: 3.455, 95% CI: 1.695-7.041.
^{e}P = 0.048, OR: 2.912, 95% CI: 1.023-8.290. ^{f}P = 0.0029, OR: 4.462, 95% CI: 1.720-11.573.
^{g}P = 0.03, OR: 0.1667, 95% CI: 0.232-0.860. ^{h}P = 0.017, OR: 0.1638, 95% CI: 0.034-0.807.
ⁱP = 0.035, OR: 0.1667, 95% CI: 0.033-0.860. ⁱP = 0.073, OR: 0.256, 95% CI: 0.065-1.006.
^{k}P = 0.043, OR: 3.103, 95% CI: 1.059-9.093. ^{l}P = 0.0029, OR: 4.462, 95% CI: 1.720-11.573.
The indicated P values for differences in the frequency of alleles and genotypes between study subgroups and fertile controls were obtained from data analysis with Fisher's Exact test.

### Example 3 References

1. Regan L. B M J. 1991; 302: 543-544.

2. Hill J A. in Creasy R, Resnik R, editors. Maternal-Fetal Medicine. 4^{th} Edition. W B Saunders Co, 1998. p423-443.

3. Wegmann T G, et al., Immunol. Today 1993; 14: 353-356.

4. Raghupathy R. Immunol. Today 1997; 18:478-482.

5. Hill J A, et al., JAMA 1995; 273: 1933-1936.

6. Raghupathy R, et al., Cell. Immunol. 1999; 196:122-130.

7. Piccinni M-P, et al., Nature Med.1998; 4:1020-1024.

8. Hill J A, et al., Am. J. Obstet. Gynecol. 1992; 166: 1044-1052.

9. Yui J, et al., Placenta 1994; 15:819-827.

10. Khan-Hanjani A, et al., Lancet 2000; 356:820-825.

11. McGuire W, et al., Nature 1994; 371: 508-510.

12. Nemetz A, et al., Immunogenetics 1999; 49: 527-531.

13. Wilkinson R J, et al., J. Exp. Med. 1999; 189: 1863-1873.

14. El-Omar E M, et al., Nature 2000; 404: 398-402.

15. Robertson S A, et al., Crit. Rev. Immunol. 1994; 14: 239-292.

16. Lichtman A H, et al., Proc. Natl. Acad. Sci. U.S.A. 1988; 85:9699-9703.

17. Manetti R., et al., Res. Immunol. 1994; 145: 93-100.

18. Oriss T B, et al., J Immunol. 1997; 158:3666-3672.

19. Huber M. et al., Int. Immunol. 1996; 8: 1257-1263 (1996).

20. Hunter C A, et al., J. Immunol. 1995; 155: 4347-4354.

21. Tominaga K, et al., Int Immunol 2000; 12:151-160.

22. Simon C, et al., Fertil Steril 1998; 70: 896-906.

23. Librach C L, et al., J. Biol. Chem. 1994; 269:493-500.

24. Von Wolff M, et al., Mol. Hum. Reprod. 2000; 6: 627-634.

25. di Giovine F S, et al., Hum. Mol. Genet. 1993; 1:450.

26. Pociot F, et al., Eur. J. Clin. Invest. 1992; 22: 396-402.

27. Unfried G, et al., Fertil. Steril. 2001; 75:683-687.

28. Yamada H, et al., Am. J. Obstet. Gynecol. 1994; 170:1339-1344.

29. Berkowitz R S, et al., Gynecol Oncol 1985; 1:70-77.

30. Berkowitz R S, et al., Gynecol Oncol 1988;29:94-100.

31. Faas M, et al., Fertility Steril. 2000; 74:1008-1013.

32. Canon J G, et al., Science 1985; 227:1247-1249.

33. Al-Harthi L, et al., J. interferon Cytokine Res. 2000; 20:719-724.

34. Santtila S, et al., Scand. J. Immunol 1998; 47: 195-198.

35. Guasch J F, et al., Cytokine 1996; 8: 598-602.

### Examples 2 and 3: Primer and Restriction Enzyme Summary

CD46 primers for amplification of and detection of polymorphism in intron 1 region of the CD46 gene:

| | | |
|---|---|---|
| 5′ primer | CACTTTCTCTAGGTTTCATACCTG | (SEQ ID NO:22) |
| 3′ primer | AGAGACCCTGTCTCAAAACAAAACA | (SEQ ID NO:23) |

Digestion with restriction enzyme HindIII: Digestion of PCR products yielded a 200 bp and a 60 bp HindIII fragment for allele 1, and produced a 260 bp fragment for allele 2. Production of all these three fragments was defined as heterozygotes for the two alleles, while production of a 200 bp and a 60 bp fragment or of a 260 bp fragment alone refers to homozygous for allele 1 or 2, respectively.

The HindIII restriction fragment length polymorphism is described in detail in the prior art (See, e.g., N. Bora, et al., J. Immunol. 146(8):2821-2825 (1991). As noted in that reference, the HindIII site (AACTT, SEQ ID NO:24) is present in the “U phenotype” product, whereas in the “L phenotype” DNA fragment there is a point mutation. In the L phenotype DNA, the “C” of the HindIII site is replaced with a “G”. This polymorphism is located in intron 1 of the CD46 gene (also referred to as “membrane cofactor protein” or “MCP”) between exon 1 (codes for 5′ UT/signal peptide) and exon 2 (codes for the first short consensus repeat).

IL1B-511 primers for amplification and detection of polymorphism:

| | | |
|---|---|---|
| 5′ primer | TGGCATTGATCTGGTTCATC | (SEQ ID NO:14) |
| 3′ primer | GTTTAGGAATCTTCCCACTT | (SEQ ID NO:15) |

Digestion is with restriction enzyme AvaI to yield:

−511T 305 bp

−511C 191 bp, 114bp.

IL1B-31 primers for amplification and detection of polymorphism:

| | | |
|---|---|---|
| 5′ primer | TCATAGTTTGCTACTCCTTGC | (SEQ ID NO:16) |
| 3′ primer | CAAAAAGCTGAGAGAGGAGGC | (SEQ ID NO:17) |

Digestion is with restriction enzyme Alu I to yield:

−31C 340bp, 80 bp

−31T 240 bp, 100 bp, 80 bp.

The digestion fragments can be detected in accordance with methods known in the art.

The complete sequence of the human gene for prointerleukin 1 beta (IL-1beta) is provided in GenBank Accession No.: X04500 (SEQ ID NO: 1) and in Clark, B. D., et al., Nucleic Acids Res. 14(20):7897-7914 (1986). IL-1 beta polymorphisms are described in the art (See, e.g., Guasch, J. F., et al., Cytokine 8(8):598-602 (1996) and di Giovine, F. S., Human Molecular Genetics 1(6):450 (1993)).

It will be understood that various modifications may be made to the embodiment disclosed herein. Therefore, the above description should not be construed as limiting, but merely as exemplifications of preferred embodiments. Those skilled in the art will envision other modifications within the scope of the invention as disclosed herein.

All references, patents, and patent applications disclosed herein are incorporated by reference in their entirety.

What is claimed is:

1. A method for evaluating a risk of recurrent pregnancy loss in a subject suspected of having immunologic reproductive failure, comprising: testing a biological sample obtained from the subject for the presence of: (1) a variant in the IL-1 beta promoter region and (2) a variant in the CD46 gene intron 1 region, wherein the presence of the variant in the IL-1B beta promoter region and the variant in the CD46 gene intron 1 region is indicative of an elevated risk of developing recurrent pregnancy loss.

2. The method of claim 1, wherein the variant in the IL-1 beta promoter region is a polymorphism of the IL-1 beta gene at position-511.

3. The method of claim 1, wherein the variant in the IL-1 beta promoter region is a polymorphism of the IL-1 beta gene at position-31.

4. The method of claim 1, wherein the variant in the CD46 gene intron 1 region is a polymorphism in the CD46 gene intron 1 region in the HindIII restriction site.

5. A method for evaluating a risk of recurrent pregnancy loss in a subject suspected of having immunologic reproductive failure, comprising: testing a biological sample obtained from the subject for the presence of: (1) a variant in the IL-1 beta promoter region and/or (2) a variant in the CD46 gene intron 1 region, wherein the presence of the variant in the IL-1B beta promoter region and the variant in the CD46 gene intron 1 region is indicative of an elevated risk of developing recurrent pregnancy loss.

6. The method of claim 5, wherein the variant in the IL-1 beta promoter region is a polymorphism of the IL-1 beta gene at position-511.

7. The method of claim 5, wherein the variant in the IL-1 beta promoter region is a polymorphism of the IL-1 beta gene at position-31.

8. The method of claim 5, wherein the variant in the CD46 gene intron 1 region is a polymorphism in the CD46 gene intron 1 region in the HindIII restriction site.

9. A kit for evaluating risk of recurrent pregnancy loss in a subject suspected of having immunologic reproductive failure, comprising in packaged combination:
(a) one or more reagents for testing a biological sample obtained from the subject for the presence of a variant in the IL-1 beta promoter region and/or a variant in the CD46 gene intron 1 region; and
(b) instructions for using the one or more reagents to determine the presence of a variant in the IL-1 beta promoter region and/or the presence of a variant in the CD46 gene intron I region to determine whether the mammal has a predisposition to immunologic reproductive failure.

10. The kit of claim 9, wherein the variant in the IL-1 beta promoter region is a variant of the IL-1 beta gene at position-511.

11. The kit of claim 9, wherein the variant in the IL-1 beta promoter region is a variant of the IL-1 beta gene at position-31.

12. The kit of claim 9, wherein the reagents for testing the subject for the presence of the variant in the IL-1 beta promoter region include a pair of amplification primers for a polymerase chain reaction amplification of the promoter region defining the polymorphism.

13. The kit of claim 9, wherein the reagents for testing the subject for the presence of the variant in the CD46 gene intron 1 region include a pair of amplification primers for a polymerase chain reaction amplification of the intron 1 region defining the polymorphism.

14. A method for treating a subject diagnosed as having recurrent pregnancy loss and suspected of having immunologic reproductive failure, comprising:
(a) selecting a subject having a variant in the IL-1B beta promoter region and/or having a variant in the CD46 gene intron 1 region; and
(b) administering to the subject an effective amount of an immunomodulating agent to prevent pregnancy loss, wherein the immunomodulating agent is selected from the group consisting of an immunomodulating agent that downregulates a T_{H}-1 immune response and an immunomodulating agent that upregulates a T_{H}-2 response.

15. The method of claim 14, wherein the immunomodulating agent is selected from the group consisting of glucocorticoids, cyclosporins, nifidipine, pentoxiphylline, progesterone and intravenous immunoglobin.
